(19) United States
(12) Patent Application Publication
(10) Pub. No.: US 2006/0024666 A1
(43) Pub. Date: Feb. 2, 2006

(54) Humanized antibodies against the venezuelan equine encephalitis virus

(76) Inventors: Shana Frederickson, Solana Beach, CA (US); Chris Hinkel, Escondido, CA (US)

Correspondence Address: FISH & NEAVE IP GROUP;ROPES & GRAY LLP, ONE INTERNATIONAL PLACE, BOSTON, MA, 02110-2624, US

(21) Appl. No.: 10/514,656

(22) PCT Filed: May 13, 2003
(86) PCT No.: PCT/US03/15083

§ 371 Date: Aug. 1, 2005

Related U.S. Application Data
(60) Provisional application No. 60/379,994, filed on May 13, 2002.

(51) Int. Cl. C12Q 1/70; C07K 16/42

(52) U.S. Cl. 435/5; 530/388.15

(57) ABSTRACT

Humanized anti-VEEV antibodies can be used to prevent and/or neutralize viral infection.

### RELATED APPLICATIONS

This application claims priority to U.S. Provisional Application Ser. No. 60/379,994 filed May 13, 2002, the entire content of which is incorporated herein by this reference.

### Government Contracts

The work described herein was funded through two SBIR contracts with the CDC (SBIR Contract Nos. 200-1999-00034 and 200-2000-10032).

### BACKGROUND

1. Technical Field

This disclosure relates to diagnostic reagents or an anti-viral compounds against the effects of Venezuelan Equine Encephalitis Virus (VEEV) infection. Specifically, humanized anti-VEEV monoclonal antibodies made in accordance with this disclosure bind to the E2^{c} epitope of the major VEEV glycoprotein (E2) and can be used to prevent and/or neutralize viral infection.

2. Background of Related Art

Venezuelan equine encephalitis (VEE) virus is an arthropod-borne alpha-virus that is endemic in northern South America, Trinidad, Central America, Mexico, and Florida (Smith et al, 1997; Phillpotts, 2002). Eight serologically distinct viruses belonging to the VEEV complex have been associated with human disease; the two most important of these pathogens are designated subtype I, variants A/B, and C. These agents also cause severe disease in horses, mules, burros and donkeys. Spread of the epizootic strains (serogroups 1 A/B and 1 C) to equines leads to a high viraemia followed by lethal encephalitis, and tangential spread to humans.

In the human host, the lymphatic system and the CNS (through direct infection by the olfactory nervous system) appear to be the universal target organs of VEEV (Smith et al, 1997). Infection results in an acute febrile syndrome and in some cases severe encephalitis. Thus, equine epizootics can lead to natural widespread outbreaks of human encephalitis involving thousands of cases and hundreds of deaths. Although infection with VEEV is usually transmitted by mosquito bite, the viruses are also highly infectious by aerosol (Smith et al., 1997). In addition to natural outbreaks, VEE virus has caused more laboratory acquired disease than any other arbovirus. Since its initial isolation, at least 150 laboratory-acquired infections resulting in disease have been reported.

The collective in vitro and in vivo characteristics of alpha-viruses lend themselves very well to weaponization: (1) readily and inexpensively produced in large amounts, (2) stable and highly infectious for humans as aerosols and (3) strains available that produce either incapacitating or lethal infection. VEEV was weaponized by the United States in the 1950's and 1960's before the U.S. offensive biowarfare program was terminated, and other countries have been or are suspected to have weaponized this agent. This virus could theoretically be produced in either a wet or dried form and potentially stabilized for weaponization. A biological warfare attack with virus disseminated as an aerosol would almost certainly cause human disease as a primary event.

Although vaccines to VEEV such as the live-attenuated TC-83 vaccine and the formalin inactivated C-84 vaccine exist, they have their disadvantages (Smith et al., 1997). Although TC-83 is solidly protective in equines and has a good safety record, it is reactogenic in up to 20% of humans, and may fail to produce protective immunity in up to 40% (Phillpotts et al, 2002). In addition, it is potentially diabetogenic and teratogenic. The inactivated VEEV vaccine, C-84, resulted in preparations that contained residual live virus and caused disease in 4% of those who received it (Smith et al., 1997). Also, the observation that hamsters given the C-84 vaccine were protected from subcutaneous challenge but not from aerosol exposure raised concerns that at-risk laboratory workers were not well protected by this vaccine. Thus, antiviral therapy with VEEV-specific human or “humanized” murine monoclonal antibodies, may offer an alternative therapy for those at risk or with a known exposure to VEEV.

Neutralizing murine antibodies have been found in studies revolving around the virulent 1A subtype Trinidad donkey parent virus and its vaccine derivative TC-83 (Mathews 1985, Roehrig 1988). Antigenic epitopes on the two virus envelope glycoproteins E1 and E2 have been determined. Eight are on the larger E2 glycoprotein and four are on the smaller E1 glycoprotein. The biological functions of hemagglutination and neutralization reside primarily on E2 and four E2 epitopes (E2^{c}, E2^{f}, E2^{g}, and E2^{h}) map to a critical neutralization domain. Passive transfer of murine antibodies that target these epitopes protected animals from a lethal virus challenge. The monoclonal antibody that was most efficient at blocking attachment of the virus to susceptible Vero or human embryonic lung cells in in vitro studies were those that defined epitopes spatially proximal to the E2^{c} epitope. The E2^{c} monoclonal antibodies were also the most efficient for neutralizing virus postattachment.

The murine antibody 3B4C-4 (Hy-4) (Mathews, 1985), binds to the E2^{c} epitope, has the isotype IgG1, and is able to mediate neutralization through bivalent binding at a critical site on the virion, as well as postattachment neutralization through Fc effector functions other than complement.

Unfortunately, in many instances, monoclonal antibodies are recognized by the human immune system as being a foreign substance not ordinarily occurring in the human body. This is referred to as immunogenicity or antigenicity in humans. For this reason, when antibodies of non-human origin are administered to humans, anti-non-human antibody antibodies are generated which result in enhanced clearance of the non-human antibodies from the body, thus reducing or completely blocking their therapeutic or diagnostic effects. Hypersensitivity reactions may also occur.

It would be to provide engineered antibodies based on antibodies from an originating species which exhibit reduced immunogenicity while maintaining an optimum binding profile that can be used to prevent and/or neutralize Venezuelan Equine Encephalitis viral infection and/or for diagnostic purposes.

### SUMMARY

Humanized antibodies and functional fragments of antibodies are described herein which bind to the E2^{c} epitope of the major VEEV glycoprotein (E2) and can be used to prevent and/or neutralize viral infection. The humanized antibodies and functional fragments of antibodies contain a complementarity determining region (specifically, CDR3) from a murine antibody produced by hybridoma cells 3B4C-4. Methods for producing the present antibodies include the steps of combining a murine CDR3 with human framework regions to provide a humanized antibody or functional fragment of an antibody.

### BRIEF DESCRIPTION OF THE DRAWINGS

FIGS. 1A and 1B show a list of murine kappa and heavy chain PCR primers used to clone the Hybridoma antibody genes.

FIG. 2 shows 3B4C-4 hybridoma sequences.

FIG. 3 shows primer sequences used for grafting the murine light chain CDR3 into a library of rearranged human kappa light chains.

FIG. 4 shows primers used to generate the chimeric heavy chain.

FIG. 5 shows the sequences of three humanized light chain clones that bind to TC-83.

FIG. 6 shows the strategy for humanization of heavy chain.

FIG. 7 shows oligo sequences used for constructing the humanized heavy chain library.

FIGS. 8A and B show the sequence for humanized clone Hy4-26.

FIG. 9A shows sequence for humanized heavy chain and 9B through E show the sequences for four different versions of humanized light chains.

FIG. 10 shows the competition curve for the humanized antibody and the murine Hy4 self competition curve.

FIG. 11 schematically shows the two step process used to clone the humanized Hy4-26A and Hy4-26C Fabs into a single vector expression system that allowed expression of the respective whole IgG1/Kappa.

FIG. 12 shows the sequence of the mammalian control cassette that was ligated into the position created when the heavy chain's bacterial control elements (ribisomal binding site and leader signal) were removed from the Fab clone.

FIG. 13 A shows the nucleic acid and amino acid sequences of the whole IgG humanized heavy chain for plasmids Hy4-26A and Hy4-26C.

FIG. 13 B shows the nucleic acid and amino acid sequences of the whole IgG humanized light chain for plasmid Hy4-26A.

FIG. 13 C shows the nucleic acid and amino acid sequences of the whole IgG humanized light chain for plasmid Hy4-26C.

FIG. 14 shows the results of ELISA screening of Hy426A-IgG and Hy426C stable cell line candidates for functional expression of IgG.

FIGS. 15A-I show the nucleic acid sequence for the vector E1m2 IgG pAPEX.

### DETAILED DESCRIPTION OF PREFERRED EMBODIMENTS

Humanized antibodies that bind to and/or neutralize VEE virus are described herein. The humanized antibodies bind to either the VEE virus itself or to a receptor involved in VEEV infection. In particularly useful embodiments, the humanized antibodies and functional fragments of antibodies described herein bind to the E2^{c} epitope of the major VEEV glycoprotein (E2) and can be used to prevent and/or neutralize viral infection. The humanized antibodies and functional fragments of antibodies preferably contain a complementarity determining region (specifically, CDR3) from a murine antibody produced by hybridoma cells 3B4C-4.

Technical and scientific terms used herein have the meanings commonly understood by one of ordinary skill in the art to which the present teachings pertain, unless otherwise defined herein. Reference is made herein to various methodologies known to those of skill in the art. Publications and other materials setting forth such known methodologies to which reference is made are incorporated herein by reference in their entireties as though set forth in full. Practice of the methods described herein will employ, unless otherwise indicated, conventional techniques of chemistry, molecular biology, Microbiology, recombinant DNA, and immunology, which are within the skill of the art. Such conventional techniques are explained fully in the literature. See, e.g., Sambrook, Fritsch, and Maniatis, Molecular Cloning; Laboratory Manual 2nd ed. (1989); DNA Cloning, Volumes 1 and 11 (D. N Glover ed. 1985); Oligonucleotide Synthesis (M. J. Gait ed, 1984); Nucleic Acid Hybridization (B. D. Haines & S. J. Higgins eds. 1984); the series, Methods in Enzymology (Academic Press, Inc.), particularly Vol. 154 and Vol. 155 (Wu and Grossman, eds.); PCR-A Practical Approach (McPherson, Quirke, and Taylor, eds., 1991); Immunology, 2d Edition, 1989, Roitt et al., C. V. Mosby Company, and New York; Advanced Immunology, 2d Edition, 1991, Male et al., Grower Medical Publishing, New York; DNA Cloning: A Practical Approach, Volumes 1 and 11, 1985 (D. N. Glover ed.); Oligonucleotide Synthesis, 1984, (M. L. Gait ed); Transcription and Translation, 1984 (Harnes and Higgins eds.); Animal Cell Culture, 1986 (R. I. Freshney ed.); Immobilized Cells and Enzymes, 1986 (IRL Press); Perbal, 1984, A Practical Guide to Molecular Cloning; and Gene Transfer Vectors for Mammalian Cells, 1987 (J. H. Miller and M. P. Calos eds., Cold Spring Harbor Laboratory); WO97/083220; U.S. Pat. Nos. 5,427,908; 5,885,793; 5,969,108; 5,565,332; 5,837,500; 5,223,409; 5,403,484; 5,643,756; 5,723,287; 5,952,474; Knappik et al., 2000, J. Mol. Biol. 296:57-86; Barbas et al., 1991, Proc. Natl. Acad. Sci. USA 88:7978-7982; Schaffitzel et al. 1999, J. Immunol. Meth. 10:119-135; Kitamura, 1998, Int. J. Hematol., 67:351-359; Georgiou et al., 1997, Nat. Biotechnol. 15:29-34; Little, et al., 1995, J. Biotech. 41:187-195; Chauthaiwale et al., 1992, Microbiol. Rev., 56:577-591; Aruffo, 1991, Curr. Opin. Biotechnot. 2:735-741; McCafferty (Editor) et al., 1996, Antibody Engineering: A Practical Approach, the contents of which are incorporated herein by reference.

Any suitable materials and/or methods known to those skilled in the art can be utilized in carrying out the methods described herein; however, preferred materials and/or methods are described. Materials, reagents and the like to which reference may be made in the following description and examples are obtainable from commercial sources, unless otherwise noted. It should be understood that the terms “including”, “included”, “includes” and “include” are used in their broadest sense, i.e., they are open ended and mean, e.g., including but not limited to, included but limited to, includes but not limited to, and include but not limited to.

Antibodies (Abs) that can be subjected to the techniques set forth herein include monoclonal Abs, and antibody fragments such as Fab, Fab′, F(ab′)₂, Fd, scFv, diabodies, antibody light chains, antibody heavy chains and/or antibody fragments derived from phage or phagemid display technologies. Functional antibody fragments are those fragments of antibodies which are capable of binding to an antigen notwithstanding the absence of regions normally found in whole antibodies. Single chain antibodies (scFv) are included in functional antibody fragments.

The antibodies or functional fragments of antibodies described herein are prepared by manipulating the sequence and structure of non-human antibodies to make them more human-like and therefore reduce or avoid immunogenicity in humans.

Several humanization strategies are known to those skilled in the art and can be used to generate the present humanized antibodies. Generally, the initial step is to obtain an initial non-human antibody known to bind to and/or neutralize VEE virus. Techniques for generating and cloning monoclonal antibodies are well known to those skilled in the art. After two or more desired antibodies are obtained, the sequence of each of the antibodies is determined, i.e., the variable regions (VH and VL) may be identified by component parts (i.e., frameworks (FRs), CDRs, Vernier zone regions and VH/VL interface regions) using any possible definition of CDRs (e.g., Kabat alone, Chothia alone, Kabat and Chothia combined, and any others known to those skilled in the art) and thus identified.

According to the present methods, one or more specific non-human antibodies are chosen based on a number of criteria including one or more of high expression and high affinity, specificity and/or activity for the VEE virus or receptors involved in VEEV infection. Screening methods for isolating antibodies with high and higher affinity for a target are well-known in the art. For example, the expression of polypeptides fused to the surface of filamentous bacteriophage provides a powerful method for recovering a particular sequence from a large ensemble of clones (Smith et al., Science, 228:1315-1517, 1985). Antibodies binding to peptides or proteins have been selected from large libraries by relatively simple panning methods, e.g., Scott et al., Science, 249:386-290, 1990; Devlin et al. Science, 249:404406, 1990; Cwirla et al., Proc. Natl. Acad. Sci. U.S.A, 87:6378-6382, 1990; McCafferty et al., Nature, 348:552-554, 1990; Lowman et al., Biochemistry, 30:10832-10838, 1992; and Kang et al., Proc. Natl. Acad. Sci. U.S.A., 88:4363-4366, 1991. A variety of techniques are known for display of antibody libraries including phage display, phagemid display, ribosomal display and cell surface display. In panning methods useful to screen antibodies, the target ligand can be immobilized, e.g., on plates, beads, such as magnetic beads, sepharose, etc., beads used in columns. In particular embodiments, the target ligand can be “tagged”, e.g., using such as biotin, 2-fluorochrome, e.g., for FACS sorting.

Screening a library of phage or phagemid expressing antibodies utilizes phage and phagemid vectors where antibodies are fused to a gene encoding a phage coat protein. Target ligands are conjugated to magnetic beads according to manufacturers' instructions. To block non-specific binding to the beads and any unreacted groups, the beads may be incubated with excess BSA. The beads are then washed with numerous cycles of suspension in PBS-0.05% Tween 20 and recovered with a strong magnet along the sides of a plastic tube. The beads are then stored with refrigeration until needed. In the screening experiments, an aliquot of the library may be mixed with a sample of resuspended beads. The tube contents are tumbled at cold temperatures (e.g., 4-5° C.) for a sufficient period of time (e.g., 1-2 hours). The magnetic beads are then recovered with a strong magnet and the liquid is removed by aspiration. The beads are then washed by adding PBS-0.05% Tween 20, inverting the tube several times to resuspend the beads, and then drawing the beads to the tube wall with the magnet. The contents are then removed and washing is repeated 5-10 additional times. 50 mM glycine-HCl (pH 2.2), 100 μg/ml BSA solution are added to the washed beads to denature proteins and release bound phage. After a short incubation time, the beads are pulled to the side of the tubes with a strong magnet and the liquid contents are then transferred to clean tubes. 1M Tris-HCl (pH 7.5) or 1M NaH₂PO₄ (pH 7) is added to the tubes to neutralize the pH of the phage sample. The phage are then diluted, e.g., 10⁻³ to 10⁻⁶, and aliquots plated with E. coli cells to determine the number of plaque forming units of the sample. In certain cases, the platings are done in the presence of XGal and IPTG for color discrimination of plaques (i.e., lacZ+ plaques are blue, lacZ-plaques are white). The titer of the input samples is also determined for comparison (dilutions are generally 10⁻⁶ to 10⁻⁹).

Alternatively, screening a library of phage expressing antibodies can be achieved, e.g., as follows using microtiter plates. Target ligand is diluted, e.g., in 100 mM NaHCO₃, pH 8.5 and a small aliquot of ligand solution is adsorbed onto wells of microtiter plates (e.g. by incubation overnight at 4° C.). An aliquot of BSA solution (1 mg/ml, in 100 mM NaHCO₃, pH 8.5) is added and the plate incubated at room temperature for 1 hr. The contents of the microtiter plate are removed and the wells washed carefully with PBS-0.05% Tween 20. The plates are washed free of unbound targets repeatedly. A small aliquot of phage solution is introduced into each well and the wells are incubated at room temperature for 1-2 hrs. The contents of microliter plates are removed and washed repeatedly. The plates are incubated with wash solution in each well for 20 minutes at room temperature to allow bound phage with rapid dissociation constants to be released. The wells are then washed multiple, e.g., 5, times to remove all unbound phage. To recover the phage bound to the wells, a pH change may be used. An aliquot of 50 mM glycine-HCl (pH 2.2), 100 μ/ml BSA is added to washed wells to denature proteins and release bound phage. After 5-10 minutes, the contents are then transferred into clean tubes, and a small aliquot of 1M Tris-HCl (pH 7.5) or 1M NaH₂PO₄ (pH 7) is added to neutralize the pH of the phage sample. The phage are then diluted, e.g., 10⁻³ to 10⁻⁶, and aliquots plated with E. coli cells to determine the number of the plaque forming units of the sample. In certain cases, the platings are done in the presence of XGal and IPTG for color discrimination of plaques (i.e., lacZ+ plaques are blue, lacZ-plaques are white). The titer of the input samples is also determined for comparison (dilutions are generally 10⁻⁶ to 10⁻⁹).

According to another alternative method, screening a library of antibodies can be achieved using a method comprising a first “enrichment” step and a second filter lift step as follows. Antibodies from an expressed combinatorial library (e.g., in phage) capable of binding to a given ligand (“positives”) are initially enriched by one or two cycles of affinity chromatography. A microtiter well is passively coated with the ligand of choice (e.g., about 10 μg in 100 μl). The well is then blocked with a solution of BSA to prevent non-specific adherence of antibodies to the plastic surface. About 10¹¹ particles expressing antibodies are then added to the well and incubated for several hours. Unbound antibodies are removed by repeated washing of the plate, and specifically bound antibodies are eluted using an acidic glycine-HCl solution or other elution buffer. The eluted antibody phage solution is neutralized with alkali, and amplified, e.g., by infection of E. coli and plating on large petri dishes containing broth in agar. Amplified cultures expressing the antibodies are then titered and the process repeated. Alternatively, the ligand can be covalently coupled to agarose or acrylamide beads using commercially available activated bead reagents. The antibody solution is then simply passed over a small column containing the coupled bead matrix which is then washed extensively and eluted with acid or other eluant. In either case, the goal is to enrich the positives to a frequency of about >1/10⁵. Following enrichment, a filter lift assay is conducted. For example, when antibodies are expressed in phage, approximately 1-2×10⁵ phage are added to 500 μl of log phase E. coli and plated on a large LB-agarose plate with 0.7% agarose in broth. The agarose is allowed to solidify, and a nitrocellulose filter (e.g., 0.45μ) is placed on the agarose surface. A series of registration marks is made with a sterile needle to allow re-alignment of the filter and plate following development as described below. Phage plaques are allowed to develop by overnight incubation at 37° C. (the presence of the filter does not inhibit this process). The filter is then removed from the plate with phage from each individual plaque adhered in situ. The filter is then exposed to a solution of BSA or other blocking agent for 1-2 hours to prevent non-specific binding of the ligand (or “probe”). The probe itself is labeled, for example, either by biotinylation (using commercial NHS-biotin) or direct enzyme labeling, e.g., with horse radish peroxidase or alkaline phosphatase. Probes labeled in this manner are indefinitely stable and can be re-used several times. The blocked filter is exposed to a solution of probe for several hours to allow the probe to bind in situ to any phage on the filter displaying a peptide with significant affinity to the probe. The filter is then washed to remove unbound probe, and then developed by exposure to enzyme substrate solution (in the case of directly labeled probe) or further exposed to a solution of enzyme-labeled avidin (in the case of biotinylated probe). Positive phage plaques are identified by localized deposition of colored enzymatic cleavage product on the filter which corresponds to plaques on the original plate. The developed filter is simply realigned with the plate using the registration marks, and the “positive” plaques are cored from the agarose to recover the phage. Because of the high density of plaques on the original plate, it is usually impossible to isolate a single plaque from the plate on the first pass. Accordingly, phage recovered from the initial core are re-plated at low density and the process is repeated to allow isolation of individual plaques and hence single clones of phage.

Screening a library of plasmid vectors expressing antibodies on the outer surface of bacterial cells can be achieved using magnetic beads as follows. Target ligands are conjugated to magnetic beads essentially as described above for screening phage vectors. A sample of bacterial cells containing recombinant plasmid vectors expressing a plurality of antibodies expressed on the surface of the bacterial cells is mixed with a small aliquot of resuspended beads. The tube contents are tumbled at 4° C. for 1-2 hrs. The magnetic beads are then recovered with a strong magnet and the liquid is removed by aspiration. The beads are then washed, e.g., by adding 1 ml of PBS-0.05% Tween 20, inverting the tube several times to resuspend the beads, and drawing the beads to the tube wall with the magnet and removing the liquid contents. The beads are washed repeatedly 5-10 additional times. The beads are then transferred to a culture flask that contains a sample of culture medium, e.g., LB+ ampicillin. The bound cells undergo cell division in the rich culture medium and the daughter cells will detach from the immobilized targets. When the cells are at log-phase, inducer is added again to the culture to generate more antibodies. These cells are then harvested by centrifugation and rescreened. Successful screening experiments are optimally conducted using multiple, e.g., rounds of serial screening. The recovered cells are then plated at a low density to yield isolated colonies for individual analysis. The individual colonies are selected and used to inoculate LB culture medium containing ampicillin. After overnight culture at 37° C., the cultures are then spun down by centrifugation. Individual cell aliquots are then retested for binding to the target ligand attached to the beads. Binding to other beads, having attached thereto, a non-relevant ligand can be used as a negative control.

Alternatively, screening a library of plasmid vectors expressing antibodies on the surface of bacterial cells can be achieved as follows. Target ligand is adsorbed to microtiter plates as described above for screening phage vectors. After the wells are washed free of unbound target ligand, a sample of bacterial cells is added to a small volume of culture medium and placed in the microtiter wells. After sufficient incubation, the plates are washed repeatedly free of unbound bacteria. A large volume, approximately 100 ml of LB+ ampicillin is added to each well and the plate is incubated at 37° C. for 2 hrs. The bound cells undergo cell division in the rich culture medium and the daughter cells detach from the immobilized targets. The contents of the wells are then transferred to a culture flask that contains about 10 ml LB+ampicillin. When the cells are at log-phase, inducer is added again to the culture to generate more antibodies. These cells are then harvested by centrifugation and rescreened. Screening can be conducted using rounds of serial screening as described above, with respect to screening using magnetic beads.

According to another embodiment, the libraries expressing antibodies as a surface protein of either a vector or a host cell, e.g., phage or bacterial cell can be screened by passing a solution of the library over a column of a ligand immobilized to a solid matrix, such as sepharose, silica, etc., and recovering those phage that bind to the column after extensive washing and elution.

One important aspect of screening the libraries is that of elution. For clarity of explanation, the following is discussed in terms of antibody expression by phage. It is readily understood, however, that such discussion is applicable to any system where the antibodies are expressed on a surface fusion molecule. It is conceivable that the conditions that disrupt the peptide-target interactions during recovery of the phage are specific for every given peptide sequence from a plurality of proteins expressed on phage. For example, certain interactions may be disrupted by acid pH's but not by basic pH's, and vice versa. Thus, variety of elution conditions should be tested (including but not limited to pH 2-3, pH 12-13, excess target in competition, detergents, mild protein denaturants, urea, varying temperature, light, presence or absence of metal ions, chelators, etc.) to compare the primary structures of the antibodies expressed on the phage recovered for each set of conditions to determine the appropriate elution conditions for each ligand/antibody combination. Some of these elution conditions may be incompatible with phage infection because they are bactericidal and will need to be removed by dialysis. The ability of different expressed proteins to be eluted under different conditions may not only be due to the denaturation of the specific peptide region involved in binding to the target but also may be due to conformational changes in the flanking regions. These flanking sequences may also be denatured in combination with the actual binding sequence; these flanking regions may also change their secondary or tertiary structure in response to exposure to the elution conditions (i.e., pH 2-3, pH 12-13, excess target in competition, detergents, mild protein denaturants, urea, heat, cold, light, metal ions, chelators, etc.) which in turn leads to the conformational deformation of the peptide responsible for binding to the target.

It should be understood that any panning method suitable for recovery of antibodies demonstrating desired characteristics (e.g. good expression and desired effect on VEEV infection) is suitable. After recovery and determination of which antibodies have the desired characteristics, the sequences of those antibodies is determined and an antibody is selected for humanization.

After the sequence has been determined, the first step in humanization a comparison is made between the sequence and one or more databases of known human antibody sequences (e.g., germline, rearranged or both). The comparison is made by aligning the sequence with sequences in the database(s) and determining the degree of homology between the sequences being compared. Computer programs for searching for alignments are well known in the art, e.g., BLAST and the like. For example, a source of human amino acid sequences or gene sequences may be found in any suitable reference database such as Genbank, the NCBI protein databank (http://ncbi.nlm.nih.gov/BLAST, VBASE, a database of human antibody genes (http://www.mrc-cpe.cam.ac.uk/imt-doc), (germline sequences), and the Kabat database of immunoglobulins (http://www.immuno.bme.nwu.edu) (rearranged sequences) or translated products thereof. If the alignments are done based on the nucleotide sequences, then the selected genes should be analyzed to determine which genes of that subset have the closest amino acid homology to the originating species antibody as described herein. It is contemplated that amino acid sequences or gene sequences which approach a higher degree homology as compared to other sequences in the database can be utilized and manipulated in accordance with the procedures described herein. In certain embodiments, an acceptable range of homology is greater than about 50%. Of course, a higher homology may be sought. In any event, the human sequences with the highest degree of homology compared with the chosen non-human antibody sequences are identified. The human sequence(s) chosen will provide at least the framework regions for the humanized antibody produced in accordance with this disclosure. At least one non-human CDR (preferably a non-human CDR3) will be positioned among these human framework regions to produce the humanized antibody in accordance with this disclosure.

It is also contemplated that more than one human sequences can be chosen to provide different portions of the humanized antibody in accordance with this disclosure. In one particularly useful embodiment, one portion of the present humanized antibody is derived from a human germline sequence and another portion of the present humanized antibody is derived from a re-arranged human antibody sequence.

The next step in constructing a humanized antibody or functional antibody fragment involves selecting CDRs to be incorporated into the framework region of the previously selected human sequence(s). The CDRs chosen can come from one or more sources. With respect to the CDR3, the CDR3 from a particular non-human antibody use in the homology determination is selected. As noted above, the particular non-human antibody from which the CDR3 is selected can be advantageously chosen based upon a number of factors including, but not limited to expression efficiency, affinity, specificity and activity with respect to VEE virus or a receptor involved in VEEV infection. Techniques for assessing each of these factors are within the purview of one skilled in the art.

With respect to CDR1 and CDR2, selection is made form one or more of the following sources. CDR1 and/or CDR2 can be selected from the particular non-human antibody use in the homology determination. Alternatively, CDR1 and/or CDR2 can be selected from the consensus sequence derived from the sequences of a plurality of antibody sequences that bind to VEE virus or a receptor involved in VEEV infection. As yet another alternative, CDR1 and/or CDR2 can be selected from the human sequences identified by comparison with the non-human antibody sequence.

The framework residues are generally assigned as acceptor species sequence except at particular positions. For example, non-homologous amino acid residues at either VH/VL interface or Vernier zone positions are maintained as a choice between donor and acceptor sequences. In general, it is desirable to keep the surface residues from the acceptor frameworks from the human antibodies to further avoid potential immunogenicity of the humanized antibody. However, some surface exposed residues are also designated as VH/VL interface or Vernier zones. In that case, choice of either donor or acceptor framework sequences is still given.

After selection and assignment of the CDRs into the acceptor framework regions, assembly of a humanized antibody or functional antibody fragment can be accomplished using conventional methods known to those skilled in the art. For example, DNA sequences encoding the altered variable domains described herein may be produced by oligonucleotide synthesis. Subsequently, nucleic acid encoding altered variable domains as described herein may be constructed by primer directed oligonucleotide site-directed mutagenesis, i.e., hybridizing an oligonucleotide coding for a desired mutation with a single nucleic acid strand containing the region to be mutated and using the single strand as a template for extension of the oligonucleotide to produce a strand containing the mutation. The oligonucleotides used for site directed mutagenesis may be prepared by oligonucleotide synthesis or may be isolated from nucleic acid encoding the target species framework by use of suitable restriction enzymes.

Any selection display system may be used in conjunction with a library according to the present disclosure. Selection protocols for isolating desired members of large libraries are known in the art, as typified by phage display techniques. Such systems, in which diverse peptide sequences are displayed on the surface of filamentous bacteriophage (Scott and Smith (1990) Science, 249: 386), have proven useful for creating libraries of antibody fragments (and the nucleotide sequences that encode them) for the in vitro selection and amplification of specific antibody fragments that bind a target antigen. The nucleotide sequences encoding the V_{H} and V_{L} regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage capsids (phagebodies). An advantage of phage- or phagemid-based display systems is that, because they are biological systems where the antibody protein is linked to its encoding gene, the selected library members can be amplified simply by growing the phage containing the selected library member in bacterial cells. Furthermore, since the nucleotide sequence that encode the polypeptide library member is contained on a phage or phagemid vector, sequencing, expression and subsequent genetic manipulation is relatively straightforward. Methods for the construction of bacteriophage antibody display libraries and lambda phage expression libraries are well known in the art (McCafferty et al. (1990) Nature, 348: 552; Kang et al. (1991) Proc. Natl. Acad. Sci. U.S.A., 88: 4363; Clackson et al. (1991) Nature, 352: 624; Lowman et al. (1991) Biochemistry, 30: 10832; Burton et al. (1991) Proc. Natl. Acad. Sci U.S.A., 88: 10134; Hoogenboom et al. (1991) Nucleic Acids Res., 19: 4133; Chang et al. (1991) J. Immunol., 147: 3610; Breitling et al. (1991) Gene, 104:147; Marks et al. (1991) J. Mol. Biol., 222: 581; Barbas et al. (1992) Proc. Natl. Acad. Sci. USA, 89: 4457; Hawkins and Winter (1992) J. Immunol., 22: 867; Marks et al., 1992, J. Biol. Chem., 267: 16007; Lerner et al. (1992) Science, 258: 1313, each of which being incorporated herein by reference).

One particularly advantageous approach has been the use of scFv phage-libraries (Huston et al., 1988, Proc. Natl. Acad. Sci U.S.A., 85: 5879-5883; Chaudhary et al. (1990) Proc. Natl. Acad. Sci U.S.A., 87:1066-1070; McCafferty et al. (1990) supra; Clackson et al. (1991) supra; Marks et al. (1991) supra; Chiswell et al. (1992) Trends Biotech., 10: 80; Marks et al. (1992) J. Biol. Chem., 267: 16007). Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys), which are incorporated herein by reference. The display of Fab libraries is also known, for instance as described in WO92/01047 (CAT/MRC) and WO91/17271 (Affymax).

Other systems for generating libraries of antibodies or polynucleotides involve the use of cell-free enzymatic machinery for the in vitro synthesis of the library members. In one method, RNA molecules are selected by alternate rounds of selection against a target ligand and PCR amplification (Tuerk and Gold (1990) Science, 249: 505; Ellington and Szostak (1990) Nature, 346: 818). A similar technique may be used to identify DNA sequences which bind a predetermined human transcription factor (Thiesen and Bach (1990) Nucleic Acids Res., 18: 3203; Beaudry and Joyce (1992) Science, 257: 635; WO92/05258 and WO92/14843). In a similar way, in vitro translation can be used to synthesize antibody molecules as a method for generating large libraries. These methods which generally comprise stabilized polysome complexes, are described further in WO88/08453, WO90/05785, WO90/07003, WO91/02076, WO91/05058, and WO92/02536. Alternative display systems which are not phage-based, such as those disclosed in WO95/22625 and WO95/11922 (Affymax) use polysomes to display antibody molecules for selection. These and all the foregoing documents also are incorporated herein by reference.

Humanized antibodies and functional antibody fragments that are cloned into a display vector can be selected against the appropriate antigen in order to identify variants that maintained good binding activity because the antibody will be present on the surface of the phage or phagemid particle. See for example Barbas III, et al. (2001) Phage Display, A Laboratory Manual, Cold Spring Harbor Laboratory Press, Cold Spring Harbor, N.Y., the contents of which are incorporated herein by reference. Although any phage or phagemid display vector would work, vectors such as fdtetDOG, pHEN1, pCANTAB5E, pRL4 or pRL5 (which are described in International Application WO 0246436A2) are useful for this methodology. For example, in the case of Fab fragments, the light chain and heavy chain Fd products are under the control of a lac promoter, and each chain has a leader signal fused to it in order to be directed to the periplasmic space of the bacterial host. It is in this space that the antibody fragments will be able to properly assemble. The heavy chain fragments are expressed as a fusion with a phage coat protein domain which allows the assembled antibody fragment to be incorporated into the coat of a newly made phage or phagemid particle. Generation of new phagemid particles requires the addition of helper phage which contain all the necessary phage genes. Once a library of antibody fragments is presented on the phage or phagemid surface, panning follows. In one embodiment, as discussed above, i) the antibodies displayed on the surface of phage or phagemid particles are bound to the desired antigen, ii) non-binders are washed away, iii) bound particles are eluted from the antigen, and iv) eluted particles are exposed to fresh bacterial hosts in order to amplify the enriched pool for an additional round of selection. Typically three or four rounds of panning are performed prior to screening antibody clones for specific binding. In this way phage/phagemid particles allow the linkage of binding phenotype (antibody) with the genotype (DNA) making the use of antibody display technology very successful. However, other vector formats could be used for this humanization process, such as cloning the antibody fragment library into a lytic phage vector (modified T7 or Lambda Zap systems) for selection and/or screening.

After selection of desired humanized antibodies and/or functional antibody fragments, it is contemplated that they can be produced in large volume by any technique known to those skilled in the art, e.g., in vitro synthesis, recombinant DNA production and the like. For example, humanized antibodies and/or functional antibody fragments may be produced by using conventional techniques to construct an expression vector encoding the humanized antibody. Those skilled in the art will readily envision suitable control sequences appropriate for expression of the antibody in a desire host.

The expression vectors may then be transferred to a suitable host cell by conventional techniques to produce a transfected host cell for expression of humanized antibodies and/or functional antibody fragments. The transfected host cell is then cultured using any suitable technique known to these skilled in the art to produce humanized antibodies and/or functional antibody fragments.

In certain embodiments, host cells may be co-transfected with two expression vectors, the first vector containing an operon encoding a heavy chain derived polypeptide and the second containing an operon encoding a light chain derived polypeptide. The two vectors may contain different selectable markers but, with the exception of the heavy and light chain coding sequences, are preferably identical. This procedure provides for equal expression of heavy and light chain polypeptides. Alternatively, a single vector may be used which encodes both heavy and light chain polypeptides. The coding sequences for the heavy and light chains may comprise cDNA or genomic DNA or both.

In certain embodiments, the host cell used to express humanized antibodies and/or functional antibody fragments may be either a bacterial cell such as Escherichia coli, or preferably a eukaryotic cell. Preferably a mammalian cell such as a chinese hamster ovary cell, CLS cells or 293EBNA, may be used. The choice of expression vector is dependent upon the choice of host cell, and may be selected so as to have the desired expression and regulatory characteristics in the selected host cell.

Once produced, the humanized antibodies and/or functional antibody fragments may be purified by standard procedures of the art, including cross-flow filtration, ammonium sulphate precipitation, affinity column chromatography, gel electrophoresis and the like.

Preferred techniques for the generation of antibodies in accordance with the present disclosure include techniques for in vitro isolation of antibody domains from animals immunized with a target antigen or antigenic fragments thereof, and selection of antibodies from synthetic libraries constructed using such domains.

RNA may be obtained from spleen and bone marrow cells of immunized mice, for example the use of Tri reagent (Molecular research center, Cincinnati, Ohio, USA). Alternative methods are known in the art and may also be used, examples of which include isolation after treating with guanidine thiocyanate and cesium chloride density gradient centrifugation (Chirgwin, J. M. et al., Biochemistry, 18, 5294-5299, 1979) and treatment with surfactant in the presence of a ribonuclease inhibitor such as vanadium compounds followed by treatment with phenol (Berger, S. L. et al., Biochemistry, 18, 5143-5149, 1979).

In order to obtain single-stranded DNA from RNA, single-stranded DNA complementary to the RNA (cDNA) can be synthesized by using the RNA as a template and treating with reverse transcriptase using oligo(dT) complementary to its polyA chain on the 3′ terminal as primer (Larrik, J. W. et al., Bio/Technology, 7, 934-938, 1989). In addition, gene specific primers can be used with oligonucleotides that bind to sequences specific to one or more antibodies (e.g., IgG1 constant region). Kits for cDNA synthesis are widely available in the art.

Specific amplification of originating species antibody variable region genes may be performed from the above-mentioned cDNA using an amplification technique such as the polymerase chain reaction (PCR). Primers such as those described in the Barbas III, et al. (2001) Phage Display, A Laboratory Manual, Cold Spring Harbor Laboratory Press, Cold Spring Harbor, N.Y., or Jones, S. T. et al., Bio/Technology, 9, 88-89, 1991, may be used for amplification of the originating species antibody variable region genes. PCR may also be performed with gene-specific primers. Single primer amplification can also be employed, such as, for example, the processes described in U.S. application Ser. Nos. 10/014,012 filed Dec. 10, 2001 and 60/323,455 filed Sep. 19, 2001, the disclosures of which are incorporated herein in their entirety. Variable region genes may be cloned into phage or phagemids, or another suitable selection system, and presented as a library for selection against a target. Library construction and panning techniques are well known in the art.

Recombinant nucleic acid including an insert coding for a heavy chain variable domain of humanized antibodies and/or functional antibody fragments fused to human constant domains γ, for example γ1, γ2, γ3 or γ4, preferably γ1 or γ4 can be constructed in accordance with the present disclosure by one skilled in the art. Likewise recombinant nucleic acid including an insert coding for light chains of humanized antibodies and/or functional antibody fragments fused to a human constant domain k or λ, preferably k an also be constructed. Additionally, recombinant nucleic acid can be constructed which codes for a recombinant polypeptide wherein the heavy chain variable domain and the light chain variable domain are linked by way of a spacer group. The nucleic acid may optionally contain a signal sequence facilitating the processing of the antibody in the host cell and/or a nucleic acid coding for a peptide facilitating the purification of the antibody and/or a cleavage site and/or a peptide spacer and/or an effector molecule. The nucleic acid coding for an effector molecule can be useful in diagnostic or therapeutic applications. Thus, effector molecules which are toxins or enzymes, especially enzymes capable of catalyzing the activation of prodrugs, may be particularly indicated. The nucleic acid encoding such an effector molecule has the sequence of a naturally occurring enzyme or toxin, or a mutant thereof, and can be prepared by methods well known in the art.

The selected CDRs are grafted into the chosen framework, for example using suitable oligonucleotides to mutate the human sequences by PCR. At the same time, variation may be introduced into the framework and/or CDR sequences. For example, FR or CDR residues which are known to affect binding site conformation may be varied. Residues which are known to be exposed are preferably mutated to match known or consensus human sequences. Antibodies with particularly favorable properties may be selected from such a library, for example using the selection procedures set forth above.

The humanized antibodies and/or functional antibody fragments may be used in conjunction with, or attached to other antibodies (or parts thereof) such as human or humanized monoclonal antibodies. These other antibodies may be catalytic antibodies and/or reactive with other markers (epitopes) characteristic for a disease against which the antibodies are directed or may have different specificities chosen, for example, to recruit molecules or cells of the target species, e.g., receptors, target proteins, diseased cells, etc. The antibodies (or parts thereof) may be administered with such antibodies (or parts thereof) as separately administered compositions or as a single composition with the two agents linked by conventional chemical or by molecular biological methods. Additionally the diagnostic and therapeutic value of the humanized antibodies and/or functional antibody fragments may be augmented by labeling the antibodies with labels that produce a detectable signal (either in vitro or in vivo) or with a label having a therapeutic property. Some labels, e.g. radionucleotides may produce a detectable signal and have a therapeutic property. Examples of radionuclide labels include ¹²⁵I, ¹³¹I, ¹⁴C. Examples of other detectable labels include a fluorescent chromosphere such as green fluorescent protein, fluorescein, phycobiliprotein or tetraethyl rhodamine for fluorescence microscopy, an enzyme which produces a fluorescent or colored product for detection by fluorescence, absorbance, visible color or agglutination, which produces an electron dense product for demonstration by electron microscopy; or an electron dense molecule such as ferritin, peroxidase or gold beads for direct or indirect electron microscopic visualization.

The humanized antibodies and/or functional antibody fragments herein may typically be administered to a patient in a composition comprising a pharmaceutical carrier. A pharmaceutical carrier can be any compatible, non-toxic substance suitable for delivery of the monoclonal antibodies to the patient, Sterile water, alcohol, fats, waxes, and inert solids may be included in the carrier. Pharmaceutically accepted adjuvants (buffering agents, dispersing agents) may also be incorporated into the pharmaceutical composition. It should be understood that compositions can contain both entire humanized antibodies and/or functional antibody fragments.

The humanized antibodies and/or functional antibody fragment compositions may be administered to a patient in a variety of ways. Preferably, the pharmaceutical compositions may be administered parenterally, e.g., subcutaneously, intramuscularly, epidurally or intravenously. Thus, compositions for parenteral administration may include a solution of the humanized antibodies and/or functional antibody fragments, or a cocktail thereof dissolved in an acceptable carrier, preferably an aqueous carrier. A variety of aqueous carriers can be used, e.g., water, buffered water, 0.4% saline, 0.3% glycine and the like. These solutions are sterile and generally free of particulate matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, etc. The concentration of humanized antibodies and/or functional antibody fragments in these formulations can vary widely, e.g., from less than about 0.5%, usually at or at least about 1% to as much as 15 or 20% by weight and will be selected primarily based on fluid volumes, viscosities, etc., in accordance with the particular mode of administration selected.

Actual methods for preparing parenterally administrable compositions and adjustments necessary for administration to subjects will be known or apparent to those skilled in the art and are described in more detail in, for example, Remington's Pharmaceutical Science, 17^{th} Ed., Mack Publishing Company, Easton, Pa. (1985), which is incorporated herein by reference.

### EXAMPLE

Cloning of Hybridoma Antibody Genes

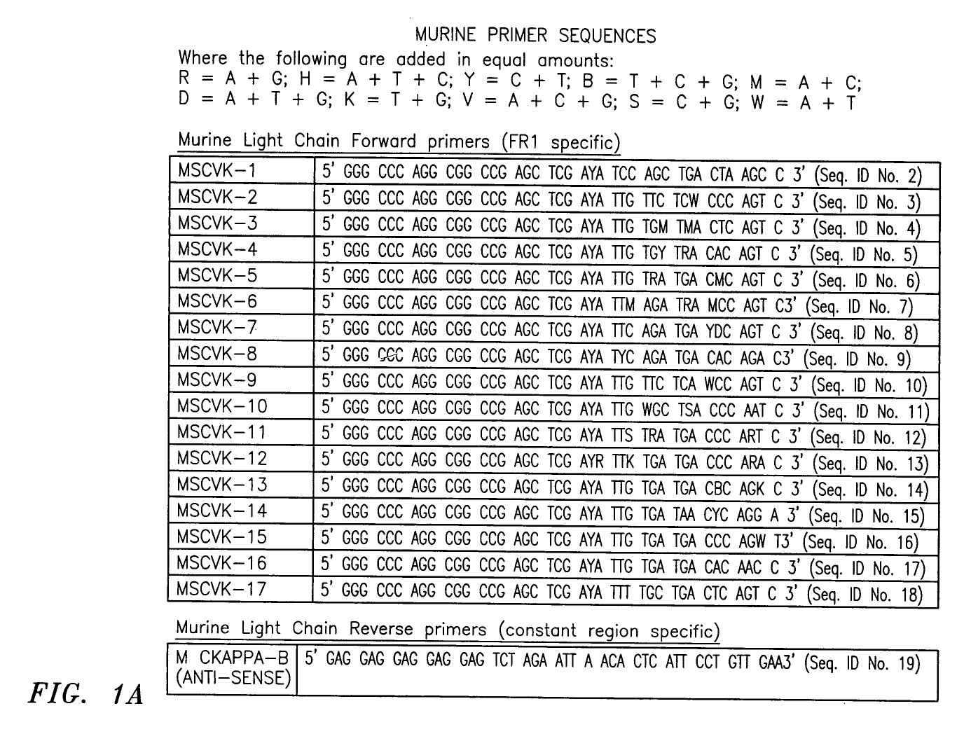

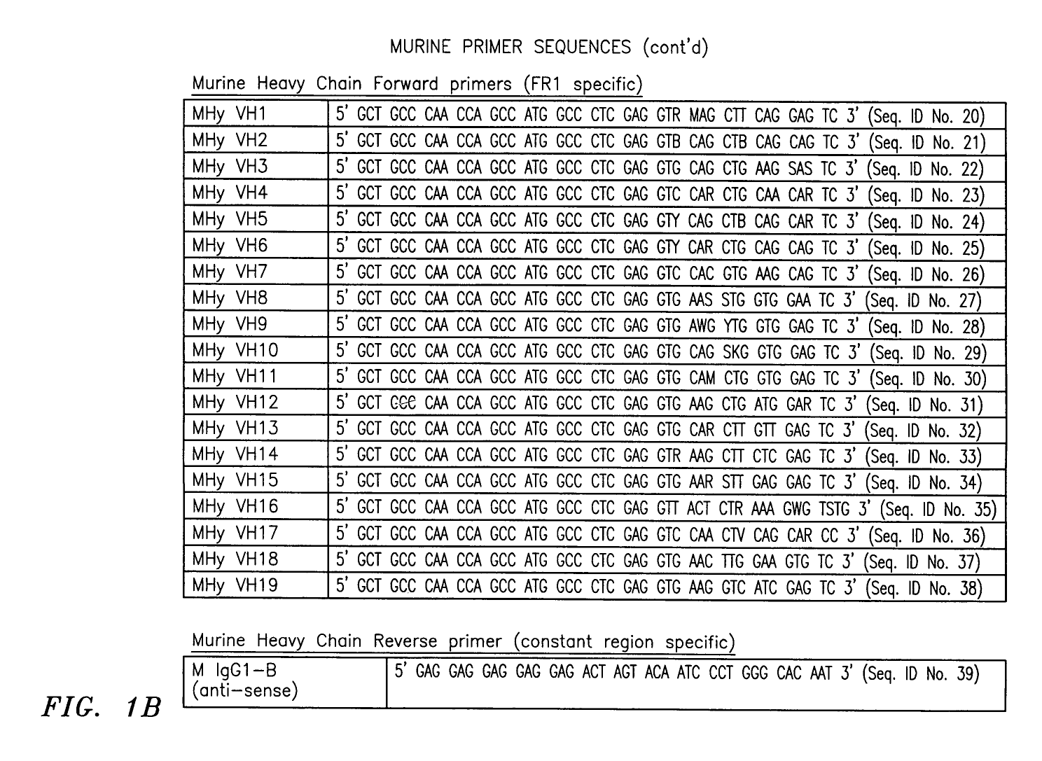

Hybridoma cells of 3B4C-4 (also called Hy-4) were harvested and added to TRI-Reagent (Molecular Research Center, MRC). The sample was processed according to MRC directions for isolation of total RNA. RNA was then used with Boehringer Manheim Biochemical First Strand cDNA kit for generation of oligo dT primed cDNA. 1 ul of the cDNA reaction was used for a 100 ul PCR reaction using Taq Polymerase (Perkin Elmer). Forward primers were pooled into three or four mixes and then used in combination with the single reverse primer for either kappa or heavy chain genes. See FIGS. 1A and 1B for a list of murine kappa and heavy chain PCR primers used. The PCR program was 94° 30′; then 30 cycles of 94° 15′, 56° 30′, 72° 1.5 minutes; 72° 10 minutes; 4° hold. A 10 ul samples of each primer pool combination was run on a 2% agarose gel to determine if the reactions worked. PCR reactions giving product were pooled and ethanol precipitated (kappa chain and heavy chain products pooled separately). After gel purification, the light chain products were digested and cloned into pRL5 by Sac I/Xba I followed by Xho I/Spe I insertion of the heavy chain products. The murine light and heavy chains in pRL5 were subjected to a couple rounds of phage display selection on TC-83 antigen to ease the identification of the correct light and heavy chain (irrelevant light chains are produced in the hybridoma cells) essentially as described previously (Radar, et. al., Proc. Natl. Acad. Sci., USA, (1998) 95, 8910-8915).

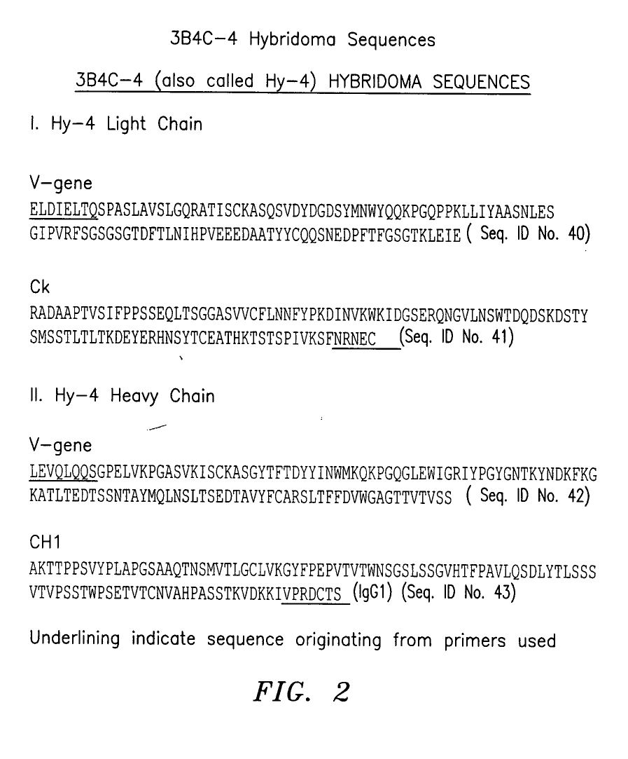

Individual clones were tested by ELISA for binding to the TC83 antigen. 200 ngs TC-83 antigen was diluted into 25 uls 0.1M NaHCO3, pH 8.6 coating buffer and applied to ½ area Costar High Bind micotiter wells. After overnight at 4° the excess antigen was washed out and the wells blocked 1 hour with BSA. Bacterial supernates were then added for 1 hour at 37° followed by 10× PBS/0.05% Tween washes. Anti-HA tag antibody 12CA5 was added and incubated 1 hour 37°. Wells were washed as above then anti-mouse IgG-Alkaline phosphatase conjugate added for 1 hour 37°. Wells again were washed as above, rinsed with water and then Sigma 104 substrate added. Color development was monitored at 405. Miniprep DNA from the positive clones were submitted for sequence analysis. Clone #78 was selected as the murine Hy-4 antibody sequence. See FIG. 2.

Humanization of the Light Chain

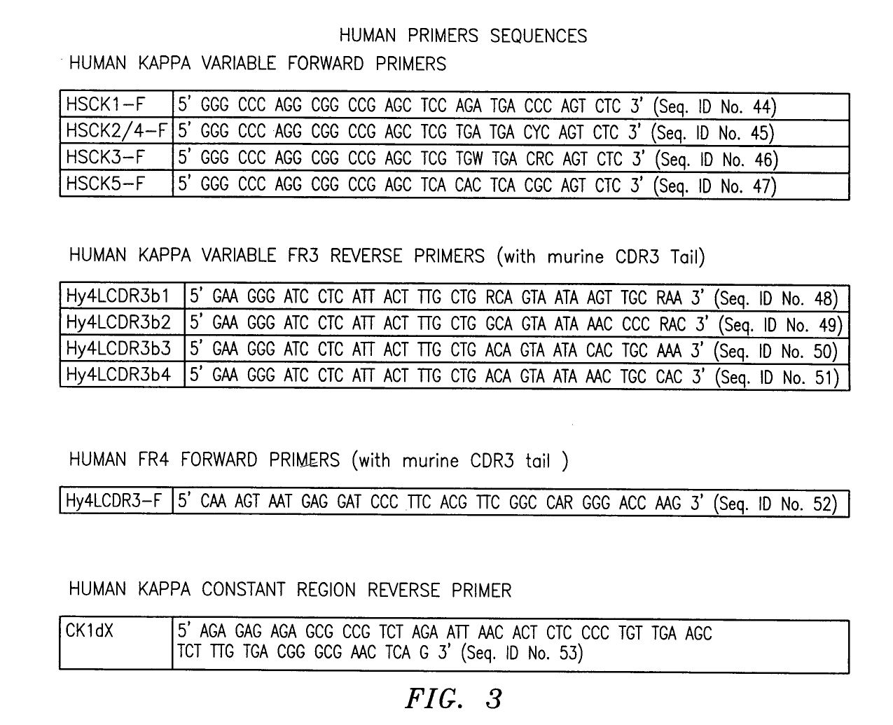

The murine light chain CDR3 was grafted into a library of rearranged human kappa light chains. Human bone marrow mononuclear cells were obtained from Poietic Technologies already treated with TRI-Reagent (Molecular Research Center, MRC). RNA was isolated and 1^{st} strand cDNA made as described above. The first PCR reactions were set up with human Kappa variable region forward primers (FR 1 specific), and reverse primers annealing at the end of FR 3. The reverse primers had a non-annealing tail of Hy-4's CDR3 sequence. See FIG. 3 for primer sequences. The product was the front half of the humanized light chain library containing a portion of the Hy-4 specific CDR3. The back half of the humanized light chain library was generated using FR4 forward primers (containing a non-annealing tail of Hy-4's CDR3) in combination with human kappa constant region reverse primer. A fusion PCR was set up using these two halves of the light chain library. The CDR3 region provides the overlap and used the same protocol as described above with RSC-F (5′ GAG GAG GAG GAG GAG GAG GCG GGG CCC AGG CGG CCG AGC TC 3′—Seq. ID No. 1) and CK1dX. The fusion PCR product was digested by Sac I/Xba I and ligated into vector pRL5 already containing the Hy-4 chimeric heavy chain (see below).

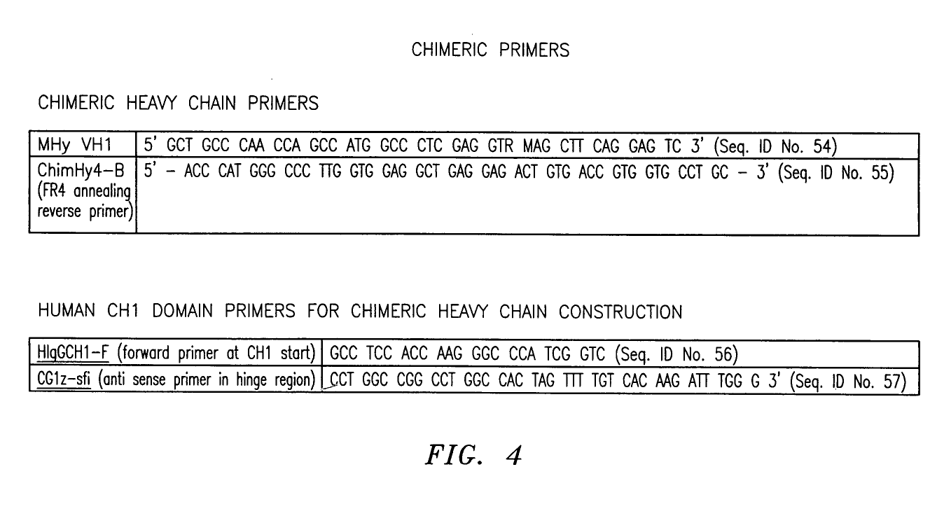

The chimeric heavy chain was generated using primers listed in FIG. 4. The murine variable region was PCR recovered from the vector DNA of murine Fab clone #78 using a murine forward primer (MHyVH1) and a chimeric heavy chain reverse primer annealing to the murine FR4 and having a tail of human CH1. The human CH1 domain was derived from a human Fab clone using the forward and reverse primers in FIG. 4. The murine VH and human CH1 domains were fused by PCR as described above using MHy-VH1 and CG1z-sfi. The fusion product was gel purified, digested by Xho I/Spe I and cloned into pRL-5.

The humanized light chain library was then subjected to four rounds of phage display selection on TC-83 antigen. Electroporation, phage amplification and panning were performed essentially as previously described (Radar, et. al., Proc. Natl. Acad. Sci. USA, (1998) 95, 8910-8915).

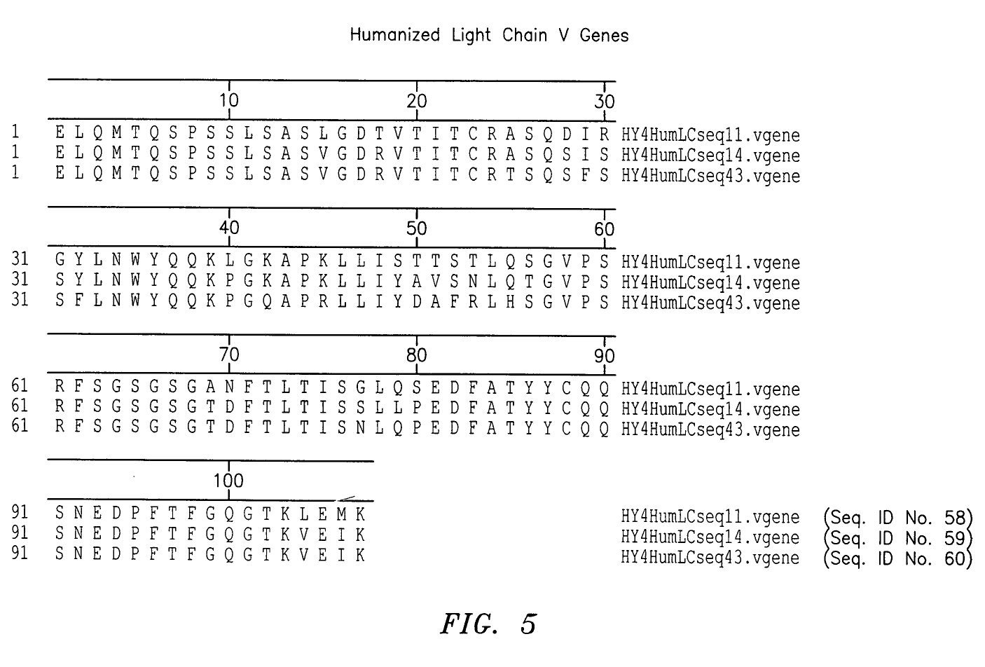

Individual clones were tested by ELISA for binding to the TC-83 following round four of panning. Three humanized light chain clones were identified as positive, Hy4-11, Hy4-14, and Hy4-43. The clones were sequenced. See FIG. 5.

Humanization of the Heavy Chain

The murine heavy chain gene was compared to nearest human germline using the VBase database. The front piece of heavy chain (amino acids prior to CDR3) were closest to the human germline variable gene DP-3. The framework 4 region (amino acids after CDR3) were closest to the human germline J-gene JH3a.

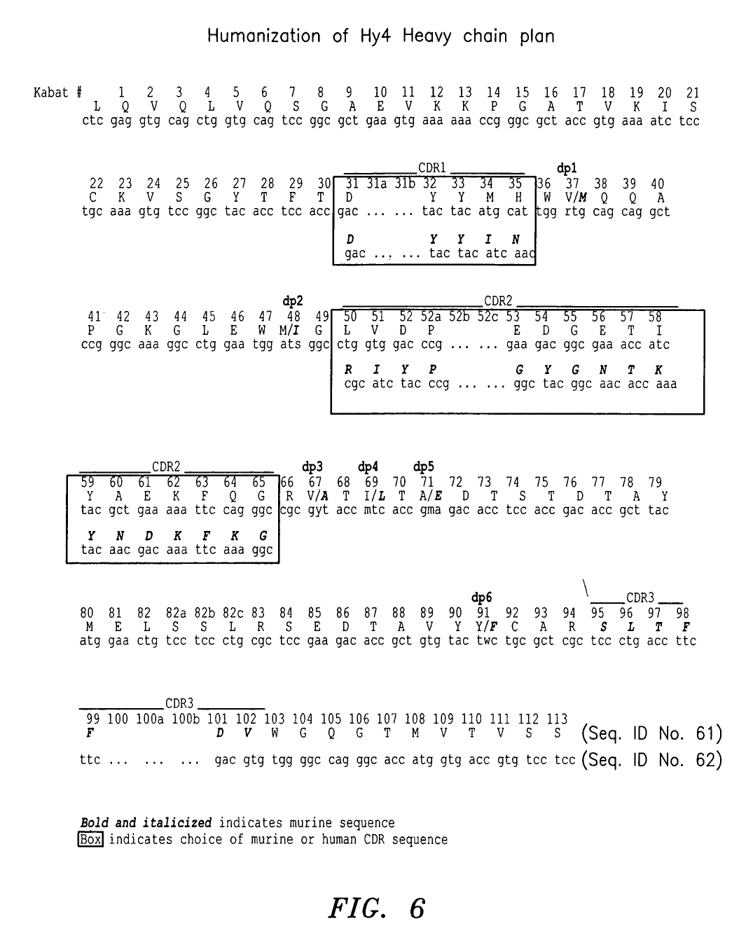

The human germline amino acids were used at all positions except 1) CDR3 was murine amino acid sequence, 2) where choice of murine or human CDR1 and CDR2 was given, or 3) where there was divergence of human and murine amino acids at selected framework positions (Vernier zone or VH/VL interface). In those cases choice between the two amino acids was given. Those degenerate positions are indicated in FIG. 6 as “dp”. The only exception to that being at the last position before CDR3 (amino acid #94 which is a Vernier zone position) where the human sequence was Thr and murine was Arg, but Arg was used since it is so predominant in rearranged human antibody sequences. Additionally, the nucleotide sequence was modified to use bacterial preferred coding.

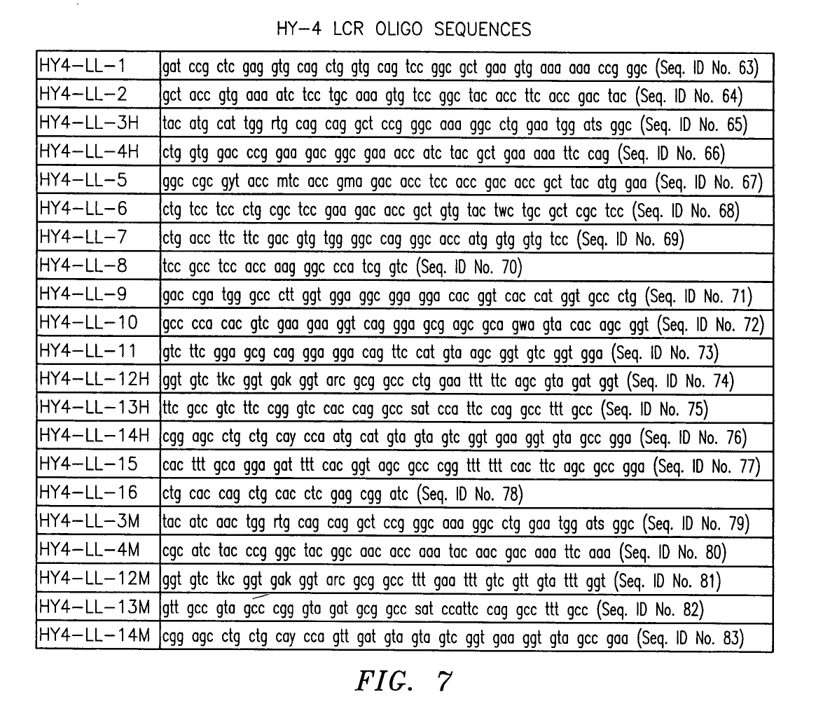

The humanized heavy chain library was constructed using oligonucleotide-ligation assembly to create separate front-halves and back-halves of the of the V-gene (the back half having a small portion of the CH 1 region for integrating the ApaI cloning site). Initially, all of the oligonucloetides were kinased on their 5′ ends with the exception of Hy4-LL-1, Hy4-LL-5, Hy4-LL-9 and Hy4-LL-13. See FIG. 7 for oligo sequences. From there, the following oligonucleotide combinations were made:

| Oligo pool | | |
|---|---|---|
| sample name | | Oligonucleotides included in pool |
| 1. | E-H | Hy4-LL-1, Hy4-LL-2, Hy4-LL-3H, Hy4-LL-4H, |
| | | Hy4-LL-13H, Hy4-LL-14H, Hy4-LL-15, |
| | | Hy4-LL-16 |
| 2. | E-M | Hy4-LL-1, Hy4-LL-2, Hy4-LL-3M, Hy4-LL-4M, |
| | | Hy4-LL-13M, Hy4-LL-14M, Hy4-LL-15, |
| | | Hy4-LL-16 |
| 3. | H-M | Hy4-LL-1, Hy4-LL-2, Hy4-LL-3H, Hy4-LL-4M, |
| | | Hy4-LL-13M, Hy4-LL-14H, Hy4-LL-15, |
| | | Hy4-LL-16 |
| 4. | M-H | Hy4-LL-1, Hy4-LL-2, Hy4-LL-3M, Hy4-LL-4H, |
| | | Hy4-LL-13H, Hy4-LL-14M, Hy4-LL-15, |
| | | Hy4-LL-16 |
| 5. | F-H | Hy4-LL-5, Hy4-LL-6, Hy4-LL-7, Hy4-LL-8, |
| | | Hy4-LL-9, Hy4-LL-10, Hy4-LL-11, Hy4-LL-12H |
| 6. | F-M | Hy4-LL-5, Hy4-LL-6, Hy4-LL-7, Hy4-LL-8, |
| | | Hy4-LL-9, Hy4-LL-10, Hy4-LL-11, Hy4-LL-12M |

These oligonucleotide combinations were separately added to reaction mixtures containing 1× ligation buffer and Ampligase Thermostable DNA Ligase (Epicentre Technologies; Madison, Wis.). The oligonucleotide ligations reactions were thermocycled with the following incubation conditions: 15 cycles of 95° C.-30 seconds, 60° C.-30 seconds, 65° C. 15 minutes and decreasing 1.0 minute/cycle; 15 cycles of 95° C.-30 seconds, 60° C.-30 seconds, 65° C.-1.0 minute; followed lastly by one final incubation at 65° C. for 15 minutes. The oligonucleotide ligation samples were gel purified on a 1.5% agarose gel. These DNA samples were then combined in the following PCR assembly reactions to create four sets of humanized Hy4 heavy chain inserts:

| Humanized Hy4 HC | 5′ DNA half fragment | 3′ DNA half fragment |
|---|---|---|
| 1. HU | E-H | F-H |
| 2. MU | E-M | F-M |
| 3. HM | H-M | F-M |
| 4. MH | M-H | F-H |

The PCR reaction mixture contained 1×PCR buffer, dNTPs, glycerol, MgCl₂ and Amplitaq Gold (Applied Biosystems; Foster City, Calif.). The PCR mixture also used the Hy4-F forward primer with a sequence of 5′-gatccgctcgaggtgcagctggt-3′ (Seq. ID No. 104) and the reverse primer, Hy4-R, with a sequence of 5′-gaccgatgggcccttggtgga-3′ (Seq. ID No. 105). The PCR reactions were thermocycled for 35 cycles of 93° C.-30 seconds, 60° C.-30 seconds and 30 seconds at 72° C., followed by a final incubation at 72° C. for 7.0 minutes. When the PCR reactions were finished, the PCR samples were electrophoresed on a 1.5% agarose gel, and the Hy4-HC DNA bands were excised from the gel and purified using the electro-elution procedure.

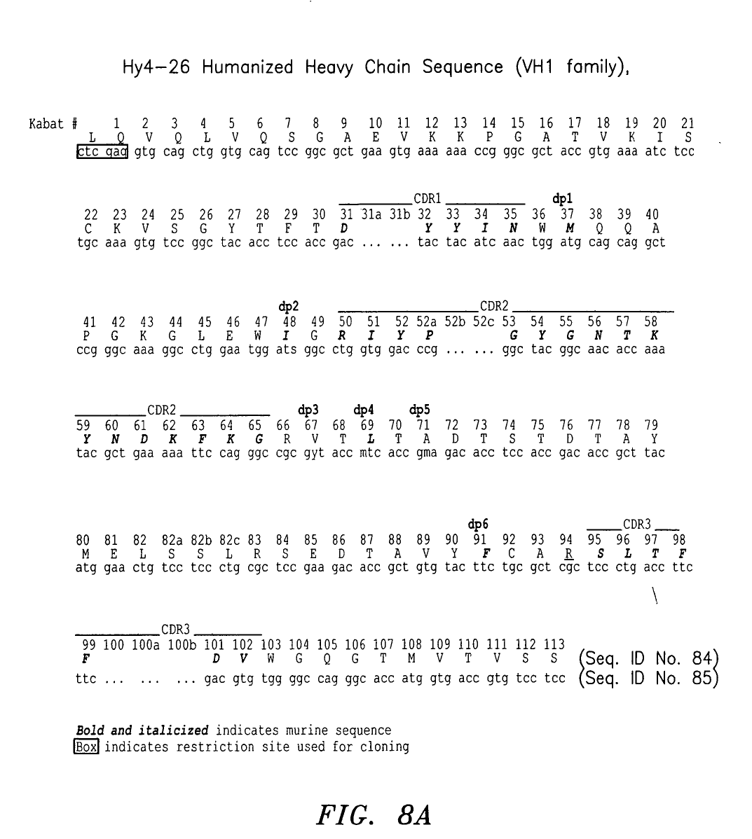

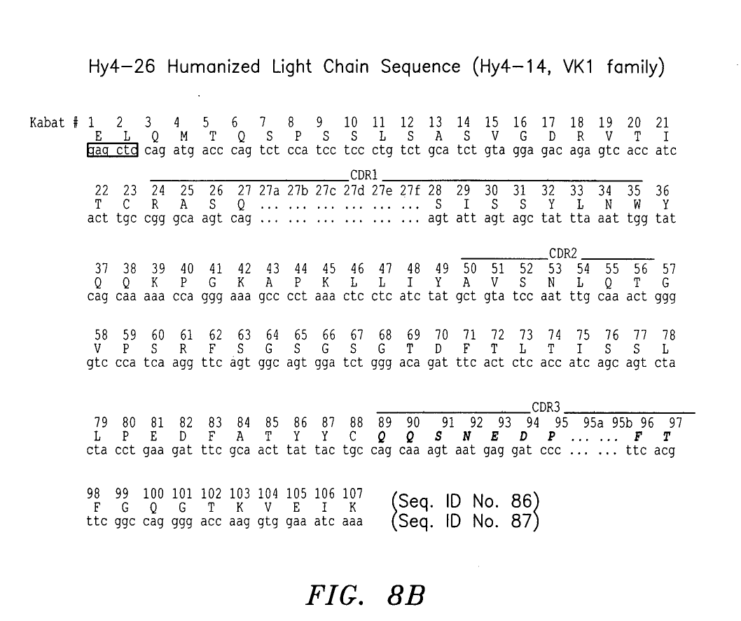

The heavy chain fragments were cloned by Xho I/Apa I into the pRL5 vector containing three Hy-4 humanized LC clones identified as described above (clones Hy4-11, Hy4-14, and Hy4-43). A phage display library was created and panned on TC-83 antigen. Following ELISA screens and sequence analysis, clone Hy4-26 was selected (contains Hy4-14 light chain). See FIG. 8.

Further Modification of the Humanized Light Chain Hy-4-14

To further improve affinity and function of the humanized Fab, murine CDR1 and CDR2 was grafted into the humanized light chain clone Hy-4-14. This was done because on germline analysis of the previously selected humanized light chains, it was discovered that all were derived from the human germline, DPK9, which was the nearest human germline to the original murine sequence. Clone Hy-4-14 had the highest homology to the germline amino acid sequence and so was selected for further modification.

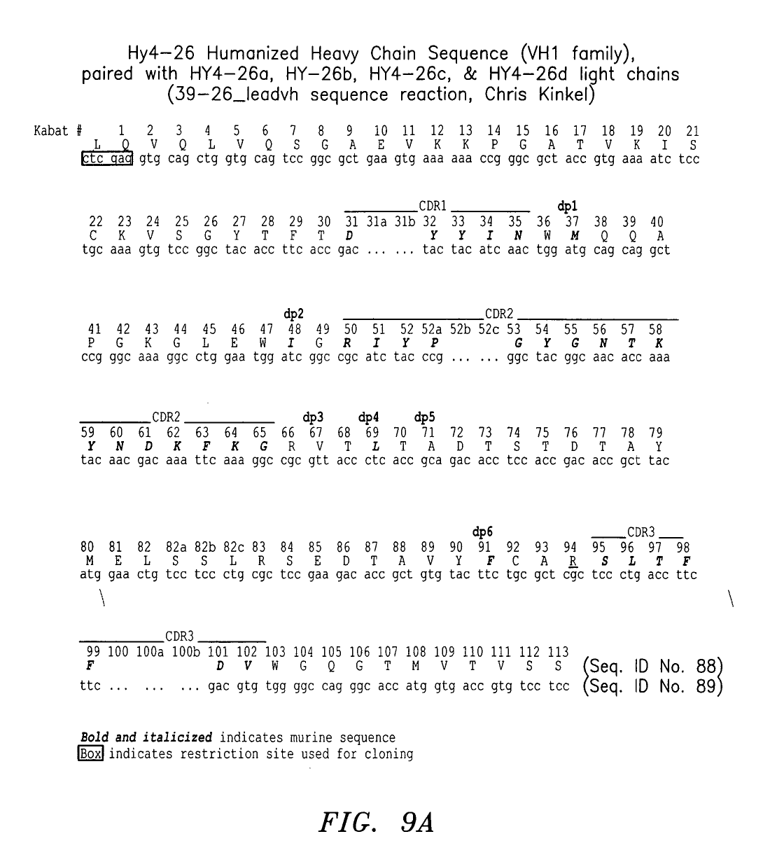

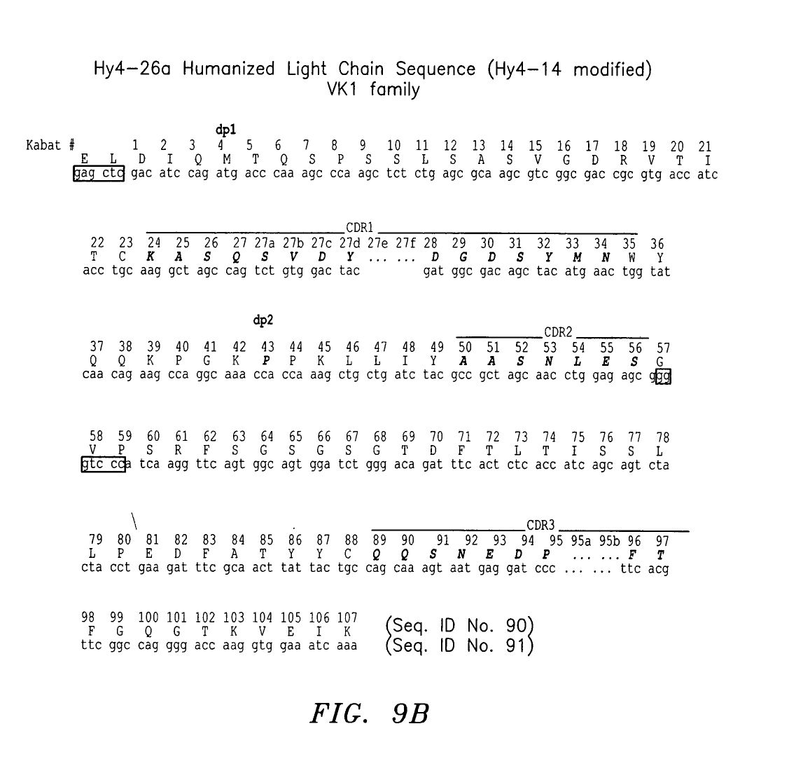

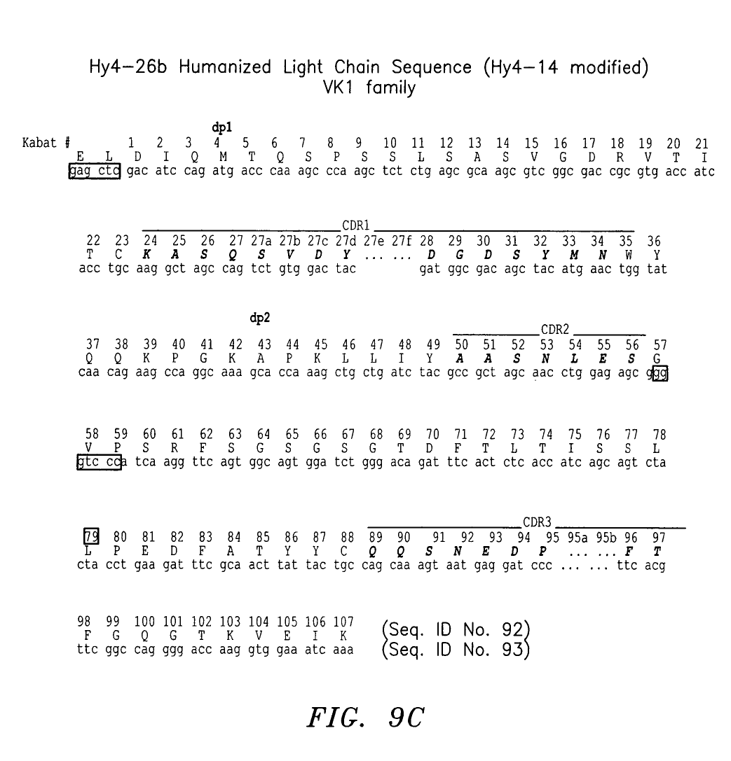

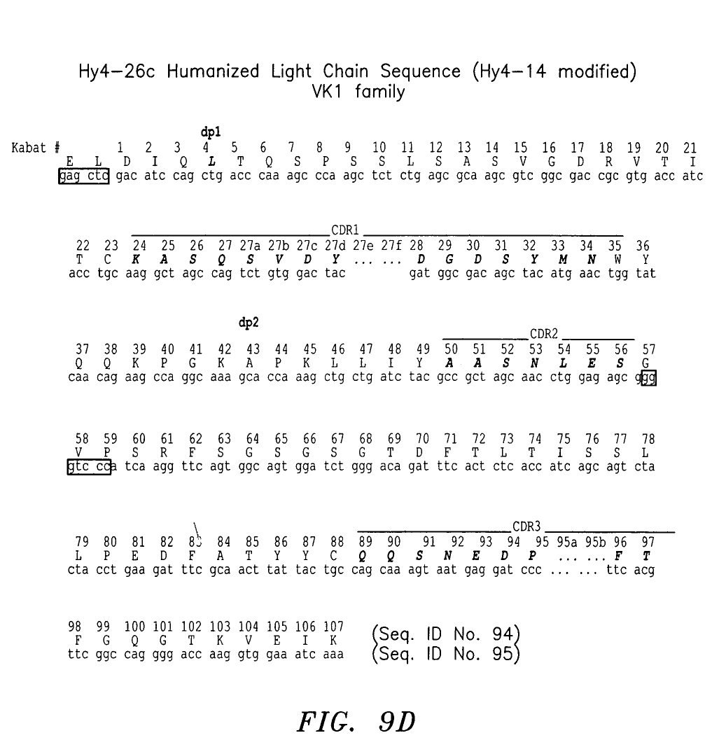

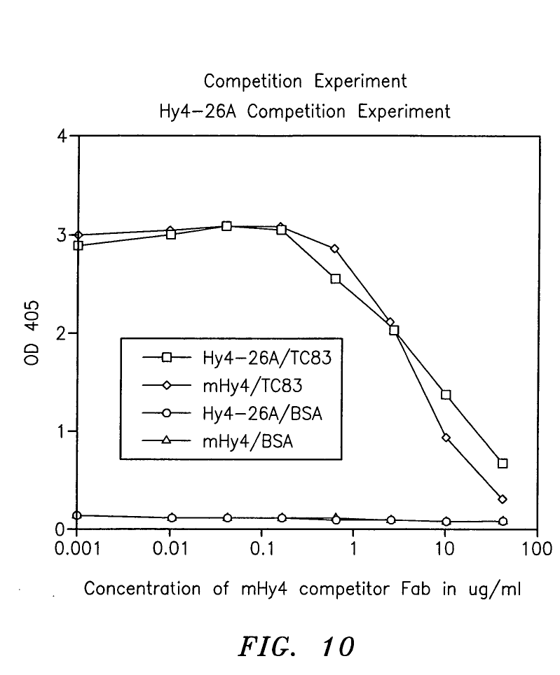

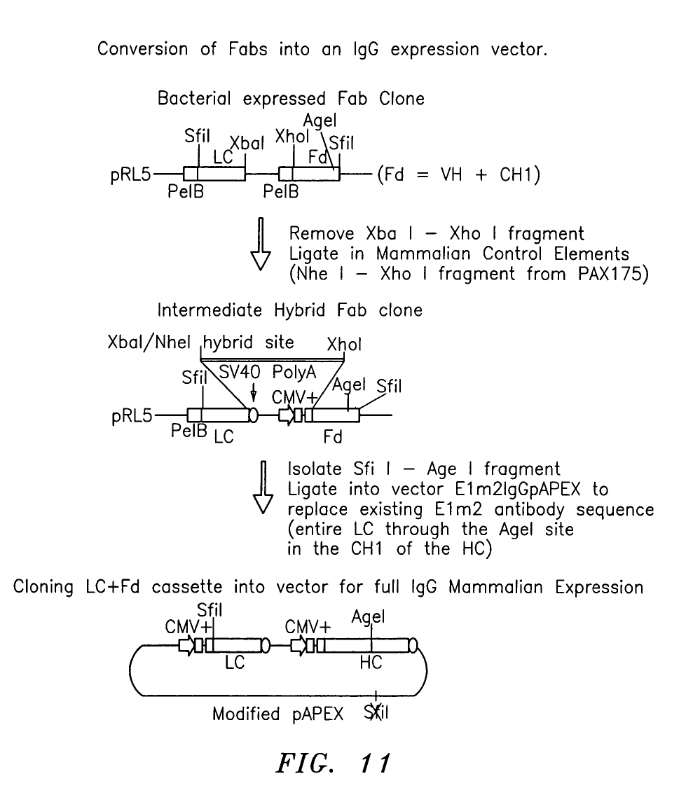

DNA cassettes for FR1 to the beginning of FR3 were created (by Aptagen, Inc., Herndon, Va.) which incorporated human germline sequence with bacterial codon preference, murine CDR1 and CDR2, as well as two positions of degeneracy where human germline and murine amino acid sequences differed in a Vernier zone and a VH/VL interface (Kabat #4 and 43, see FIG. 9). The two positions of choice resulted in there being a total of four cassettes.

The cassettes were cloned into Hy4-26 light chain by Sac I/PpuM I. The four different versions were designated as Hy4-26A, Hy4-26B, Hy4-26C, and Hy4-26D (FIG. 9). His tag purified preparations of each Fab were made using Ni-NTA spin kit (QIAGEN, Valencia, Calif.). Fabs were tested by ELISA for binding to the TC-83 antigen.

Competition of HY4-26A

The Hy4-26A Fab was compared with the original murine Hy4 Fab in a competition ELISA experiment to examine the epitope specificity of the Hy4-26A Fab. This procedure was initiated with the titration of both the Hy4-26A and mHy4 Fabs in a standard ELISA-assay. The concentrations of the Fabs used in this experiment ranged from 40 ng/μl and decreased four-fold for a strip of eight microplate wells total down to a concentration of 0.0024 ng/μl. Also, the primary detection antibody used was the 12CA5 (anti HA-tag) while the secondary antibody was the anti-mouse IgG Fc-specific alkaline-phosphatase conjugate (Sigma; St. Louis, Mich.). The titration ELISA was allowed to develop for approximately ½-hour at which point it was read in an ELISA plate reader.

Upon graphical analysis of the data from the titration ELISA experiment, a concentration was chosen for both the Hy4-26A and mHy4 Fab that represented level at which approximately 80% maximal binding occurs. This was the specific concentration that each respective Fab was used at for the competition ELISA. A modified murine Hy4 that lacked the HA immuno-tag was used as the specific competitor in the experiment. The experiment was set-up so that the Hy4-26A (and HA tagged murine Hy4) were held at the previously determined concentration and the murine Hy4 lacking the HA-tag was added in increasing amounts. The actual amounts of the competitor mHy4 began at 0.0096 ng/μl and rose four-fold in a series of seven wells to a maximal concentration of 40 ng/μl. The murine Hy4 Fab possessing a HA-tag was incorporated as a self-competition control, and wells lacking competitor murine Hy4 were used as a check for maximum signal levels. The competition ELISA was detected and developed in the same manner as the titration ELISA described above.

FIG. 10 shows that the competition curve for the humanized antibody is essentially the same as the murine Hy4 self competition curve, indicating that the Hy4-26A recognizes the same epitope on TC-83.

Conversion of Fabs into an IgG expression vector: For conversion of antibody clones into full IgGs, the coding regions for both the light and heavy chains, or fragments thereof, can be separately cloned out of the bacterial vector and into mammalian vector(s). A single vector system can be used to clone both light and heavy chain cassettes into the same plasmid. Alternatively, dual expression vectors where heavy and light chains are produced by separate plasmids can be used. Suitable expression vectors can readily be identified by those skilled in the art. Mammalian signal sequences need to be either already present in the final vector(s) or appended to the 5′ end of the light and heavy chain DNA inserts. This can be accomplished by initial transfer of the chains into a shuttle vector(s) containing the proper mammalian leader sequences. Following restriction enzyme digestion, the light chain and heavy chain regions, or fragments thereof, are introduced into final vector(s) where the remaining constant regions for IgG1 are provided either with or without introns. In some cases where introns are used, primer design for PCR amplifying the light and heavy chain variable regions out of pRL5 may need to include exon splice donor sites in order to get proper splicing and production of the antibodies in mammalian cells.

With either vector expression system (single or dual plasmid), the production of antibody light and heavy chains can be driven by promoters that work in mammalian cells such as, but not limited to, CMV, SV40, or IgG promoters. Additionally, the vector(s) will contain a selectable marker for growth in bacteria (such as, but not limited to, ampicillin, chloramphenicol, kanamycin, or zeocin resistance). Selectable markers for mammalian cells (such as, but not limited to, DHFR, GS, gpt, Neomyocin, or hygromyocin resistance) may also be present in the IgG vector(s), or could be provided on a separate plasmid by co-transfection.

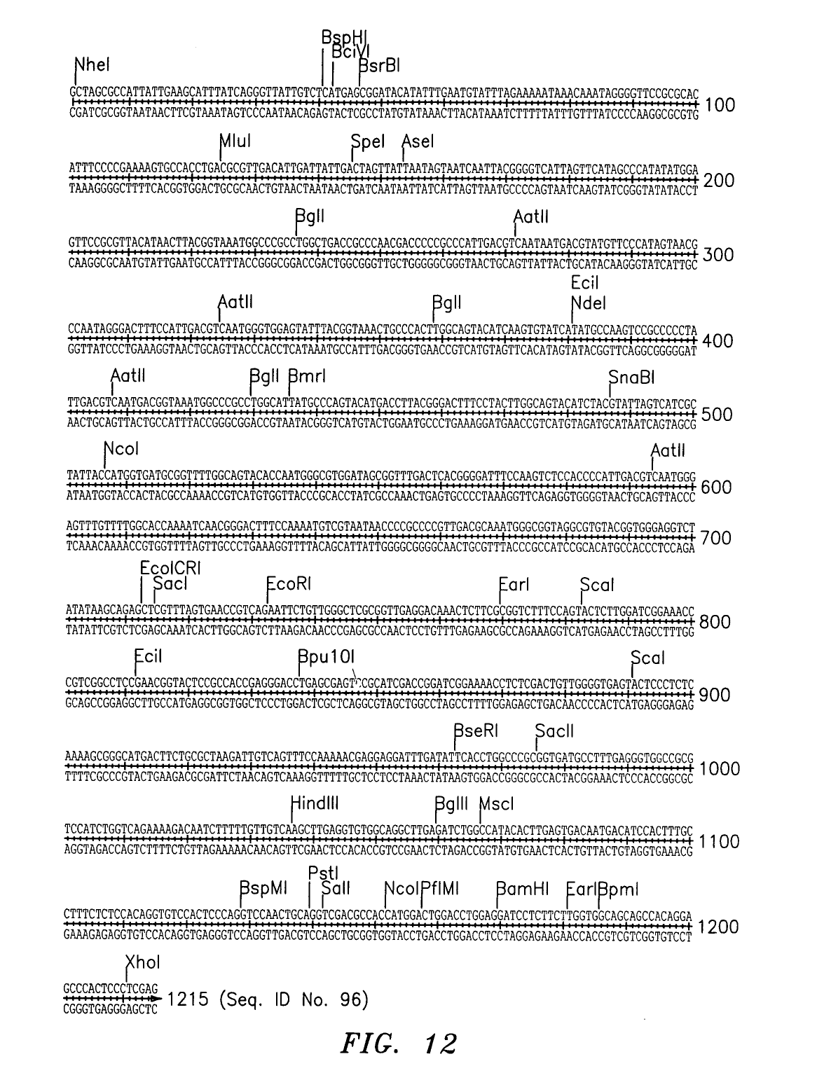

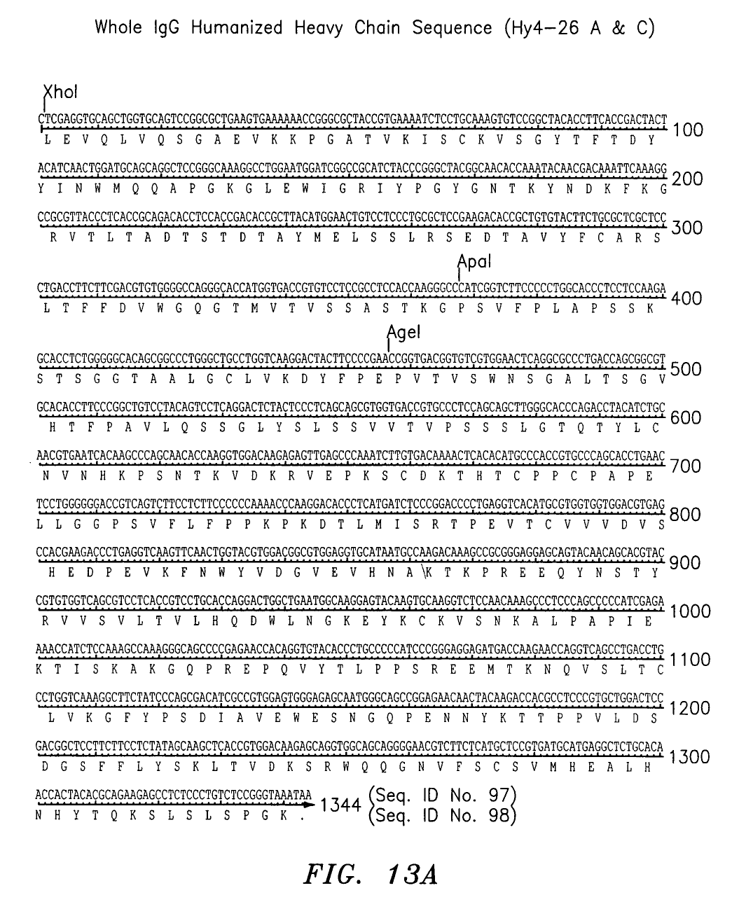

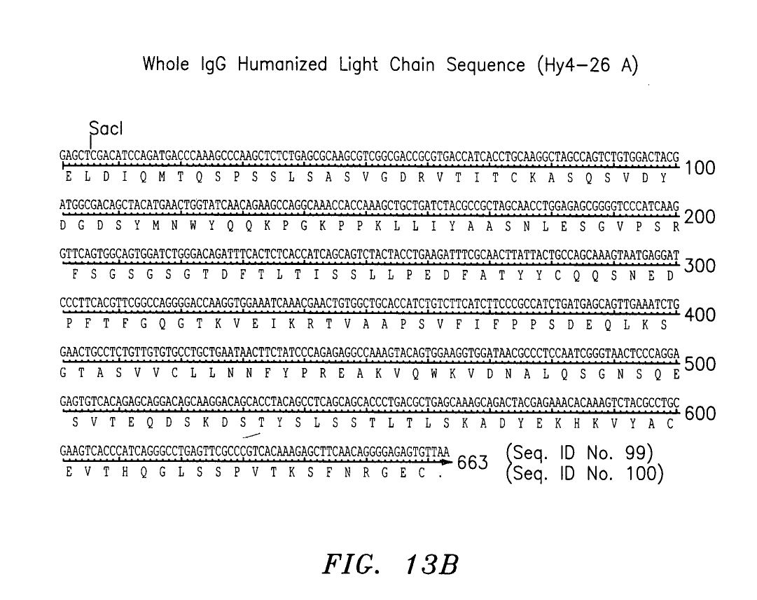

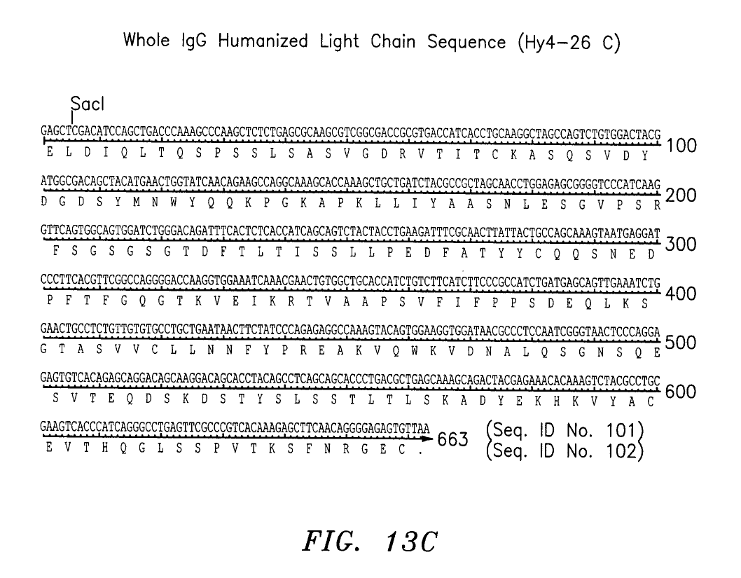

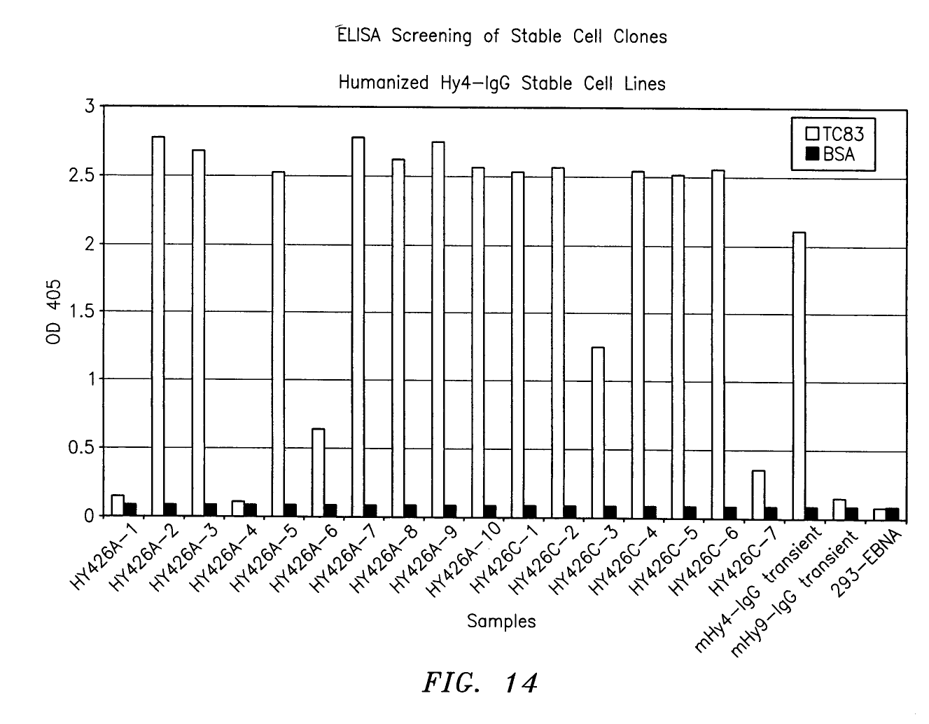

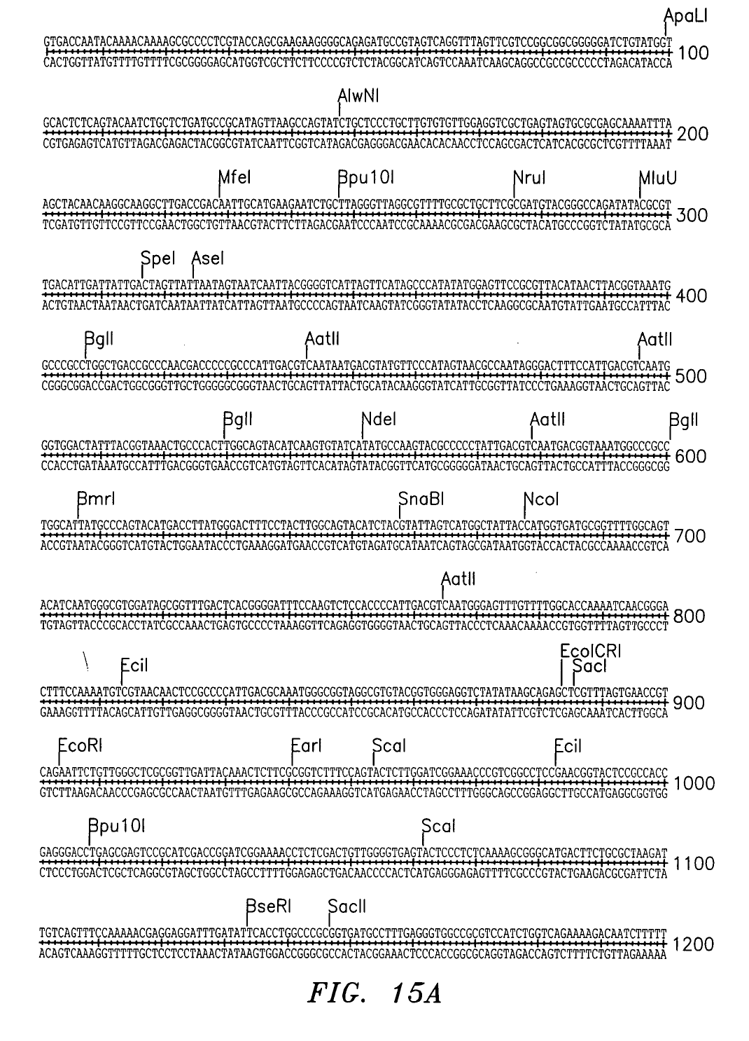

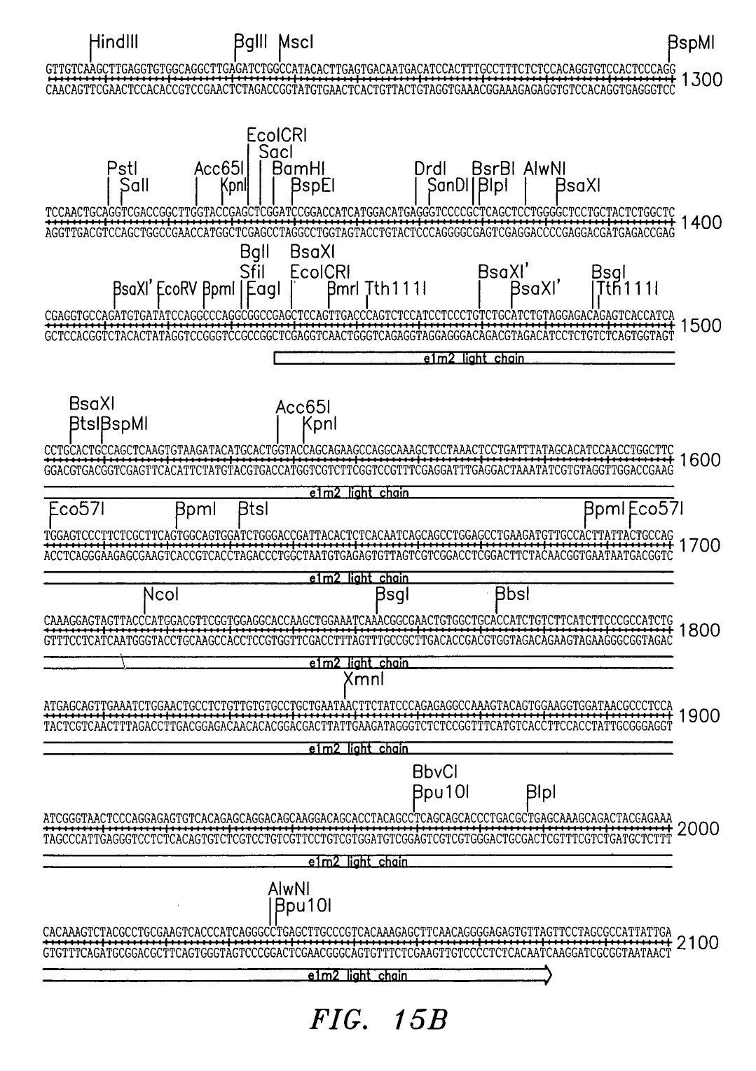

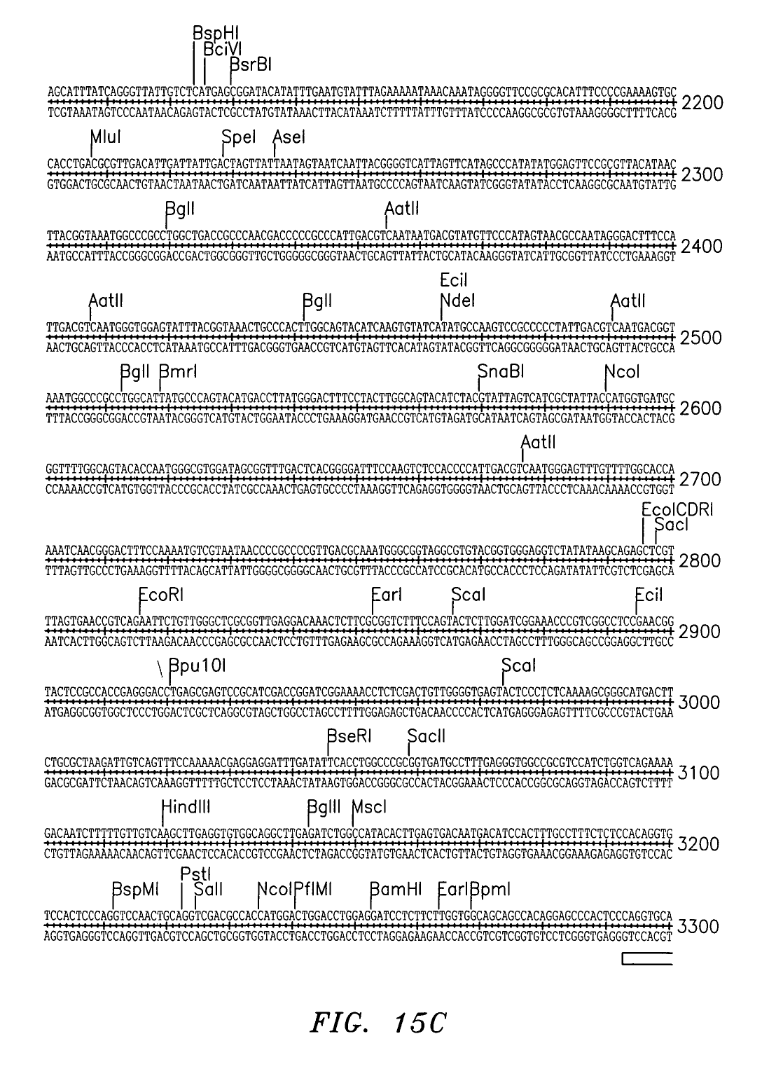

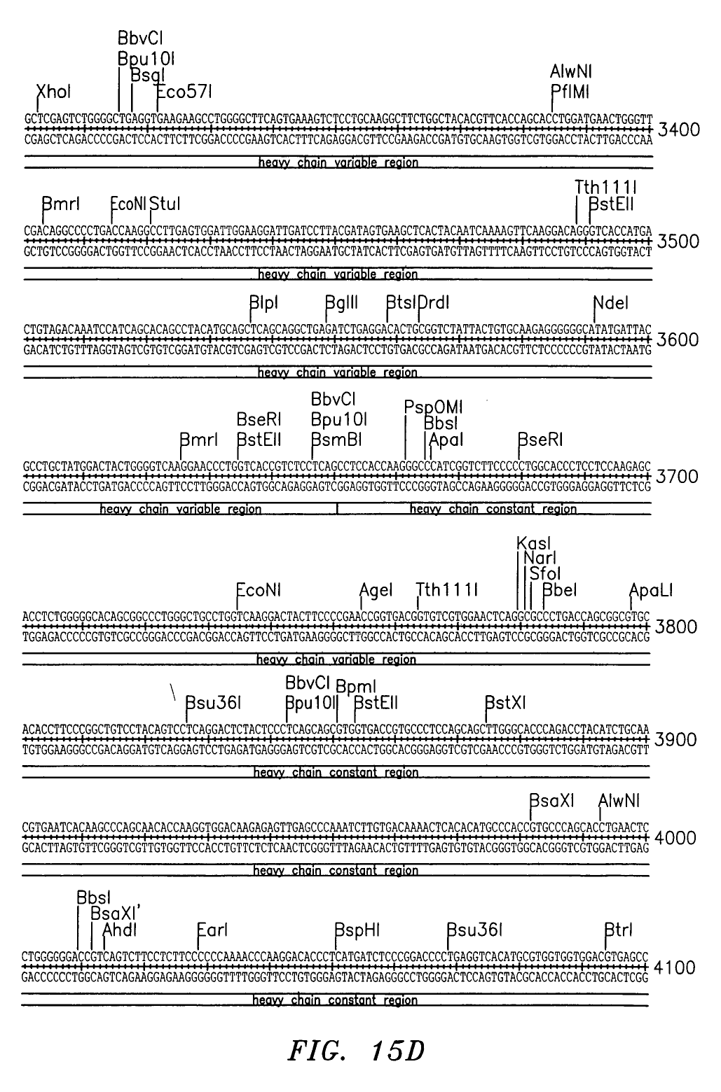

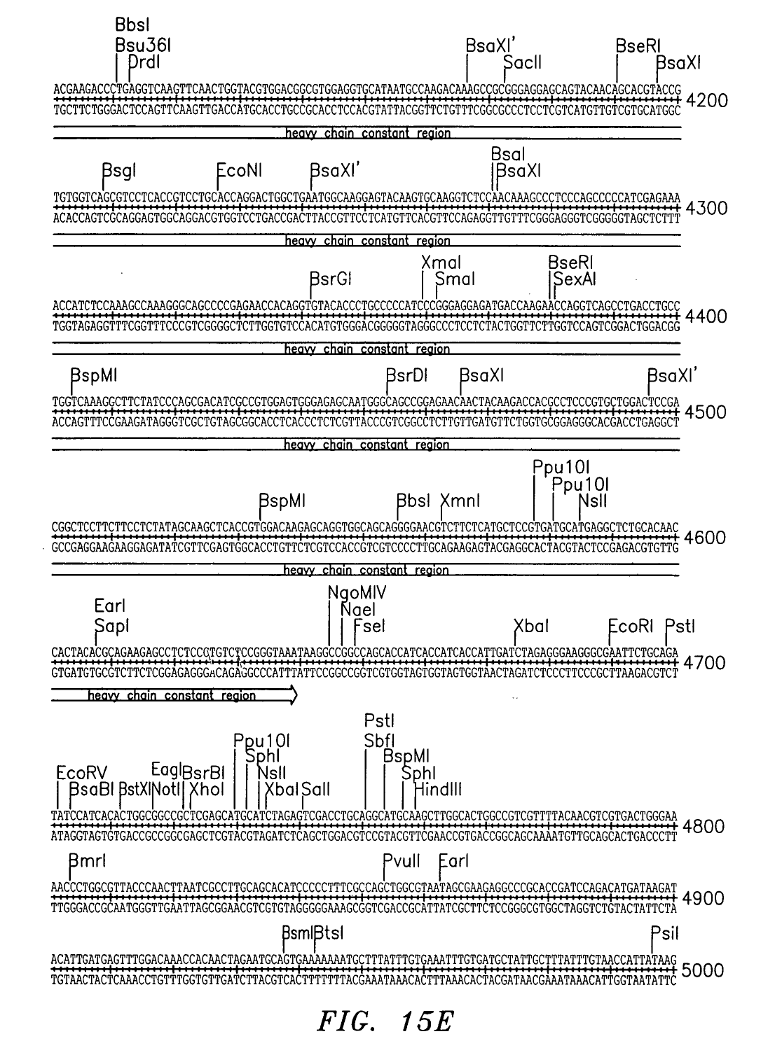

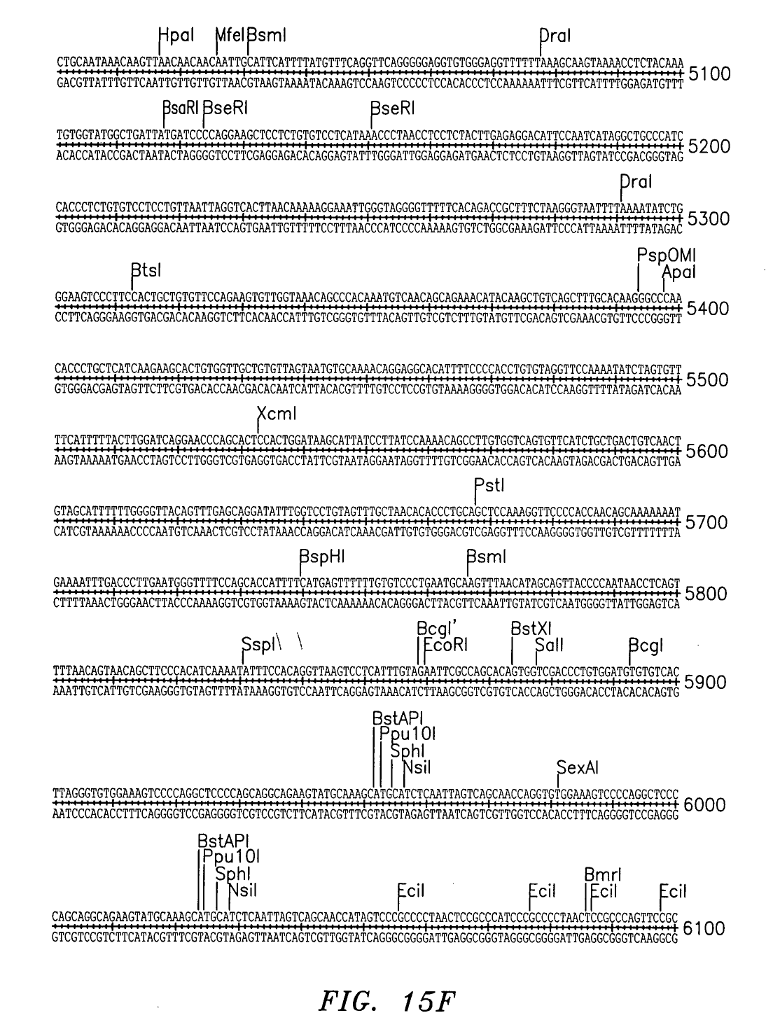

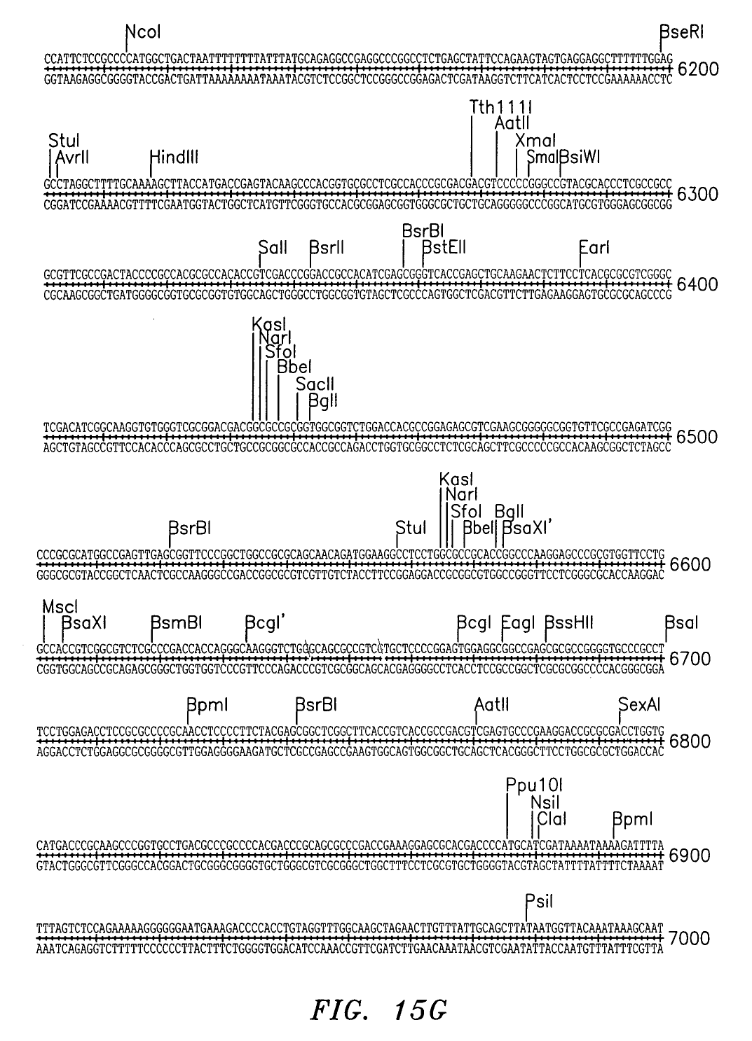

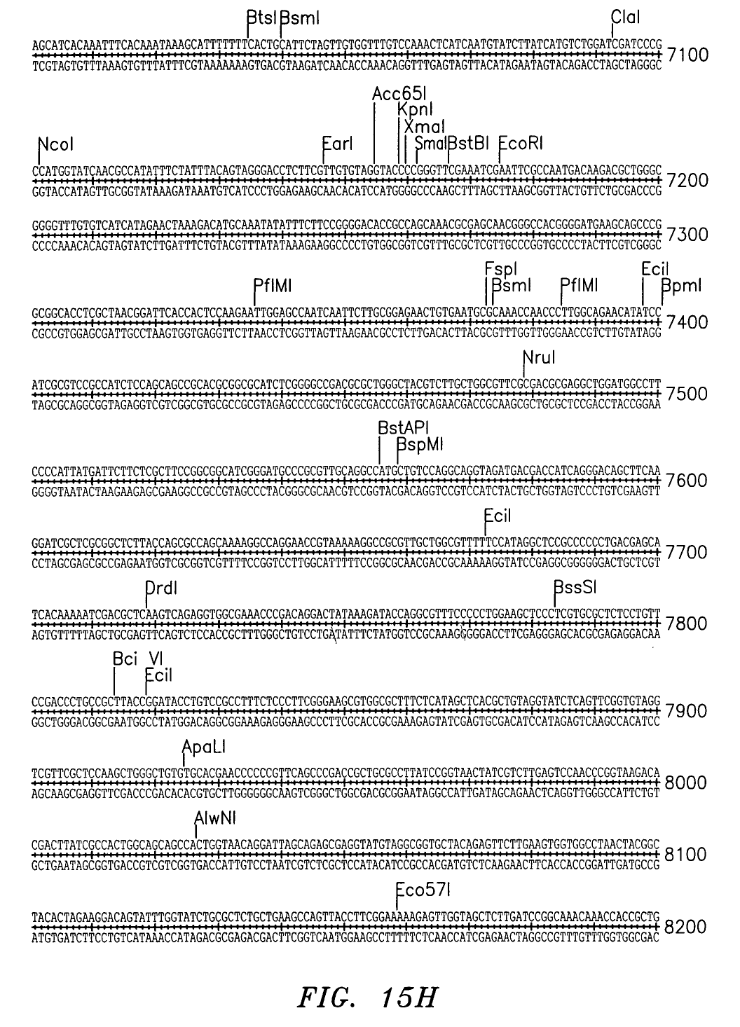

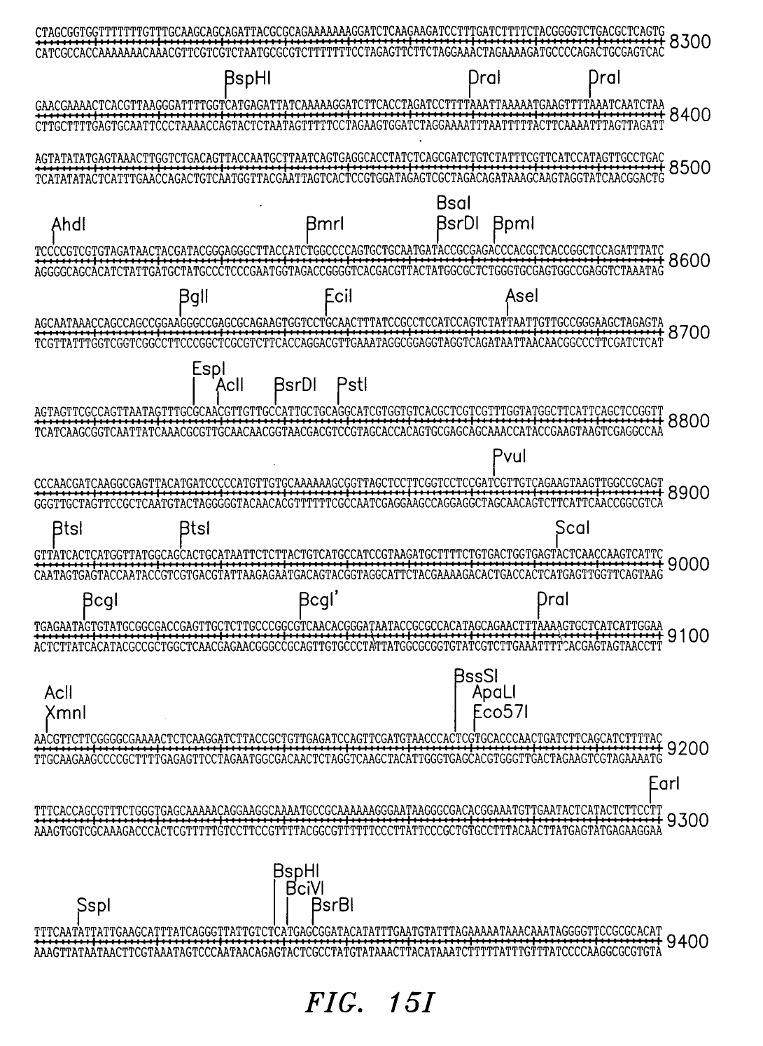

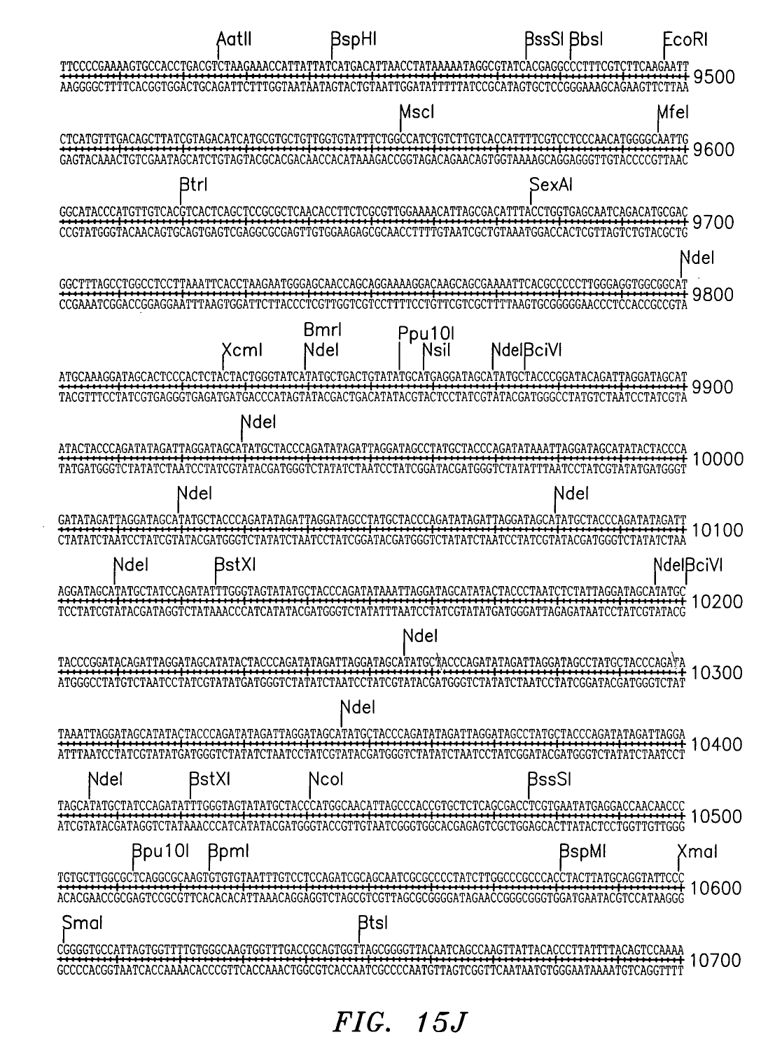

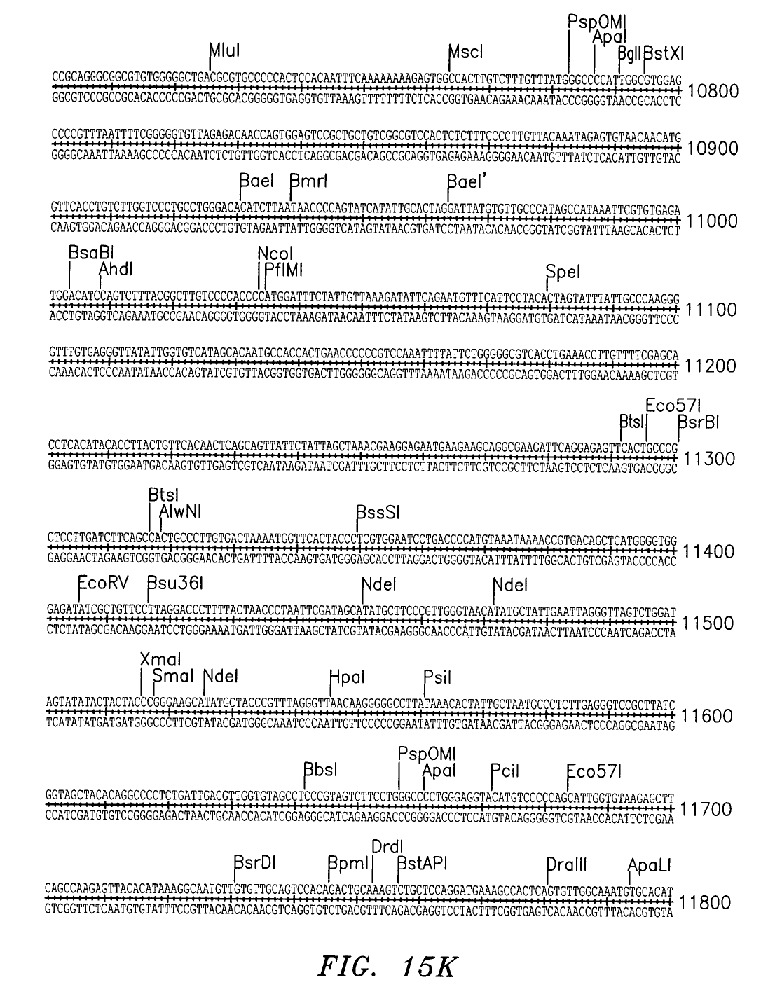

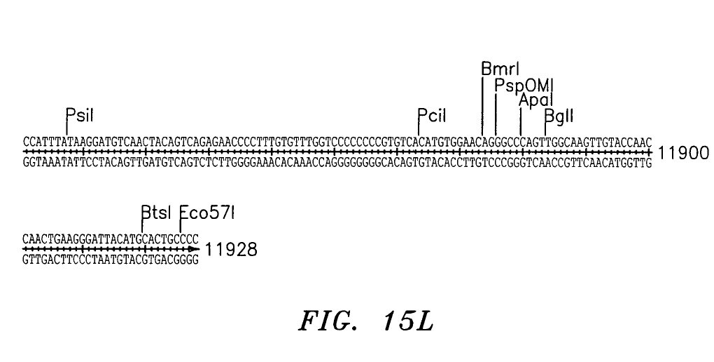

The humanized Hy4-26A and Hy4-26C Fabs were separately cloned into a single vector expression system that allowed expression of the respective whole IgG1/Kappa. The two step process (shown schematically in FIG. 11) involved first removing the heavy chain's bacterial control elements (ribisomal binding site and leader signal) from the Fab clone by digestion with Xba I and Xho I. The mammalian control element cassette (containing a poly A signal for the LC, a CMV promoter to direct HC expression and a HC leader signal) was ligated into that location Xba I and Nhe I digested DNA have compatible DNA overhangs that can be ligated together, but subsequently can not be re-cut with either restriction enzyme. The sequence of the mammalian control cassette is in FIG. 12. The resulting hybrid Fab clone had bacterial control elements in front of the LC and mammalian control elements in front of the Fd heavy chain fragment, and was therefore not for expression purposes, but an intermediate construct. The hybrid Fab clone was then digested with Sfi I and Age I to release a cassette containing the LC (without the bacterial control elements), poly A signal, CMV promoter, HC leader signal and the HC fragment up to the native AgeI site in the CH1 domain. This cassette was placed into E1m2 IgG pAPEX (the sequence of which (Seq. ID No 103 is shown in FIGS. 15A-15I) containing another CMV promoter and leader signal for expression of the LC, as well as the remaining portion of the IgG1 constant domain. DNA was sequenced to confirm construction of the desired Hy4-26A-IgG plasmid or Hy4-26C-IgG plasmid. See FIG. 13 A, B, C.

PCR reactions: Approximately 10.0 ng of vector DNA was used as the template with 1.0 ng/μl forward and reverse primer (MWG Biotech), 1.25U Taq gold (Applied Biosystems), 1×PCR buffer with 2.0 mM MgCl₂ and 7.5% glycerol in a total volume of 50 μl in each PCR tube. The PCR reactions were amplified for 30 cycles of; 93′ for 30 seconds, 60′ for 30 seconds and 72′ for 30 seconds, with a pre-incubation at 93° C. for 10.0 minutes and a final incubation at 72′ for 5.0 minutes. To generate enough PCR product for subsequent restriction digestion and cloning, 10 replicates of the PCR reaction was done for a 500 μl total combined volume. This sample Was first “cleaned-up” with the Qiagen PCR Purification kit before moving onto the next step of the cloning process.

Restriction Enzyme Digestions: For recombinant DNA cloning, 30 μg of DNA in 400 μl total volume was digested with the appropriate 1× restriction digestion buffer (New England Biolabs), 1.0 μg/ml BSA and with at least 3× excess of the desired restriction enzyme(s). The incubations lasted for approximately 2-3 hours at the specific temperature indicated for the enzymes. For diagnostic analysis of cloned samples, 5.0 μl of Qiagen miniprep purified DNA was added to 1× restriction enzyme (RE) buffer, 1.0 μg/ml BSA and 1.0 μl of each restriction enzyme. The samples were incubated for about 1-2 hours and loaded onto a 1.5% agarose/EtBr gel for the visualization of the DNA bands.

DNA fragment isolation and purification: Loading dye was added to the 400 μl RE digested sample and loaded into multiple wells of a 1.5% agarose/EtBr gel. The gel was electrophoresised for about 2.0 hrs until there was reasonable separation of the desired DNA band. The gel fragments were cut from the gel, combined into a dialysis membrane enclosure and placed back into the agarose gel box. Voltage was applied to the dialysis membrane to “eletro-elute” the DNA fragment from the agarose gel piece. The buffer surrounding the agarose gel pieces was removed from the dialysis membrane and passed through a spin filter to rid the sample of any extraneous agarose gel debris. Lastly, the DNA fragment was precipitated with 50% isopropanol and quantitated using a Picogreen DNA quantitation kit (Molecular Probes).

Ligation reactions and bacterial transformations: Ligation reactions were designed with a 3:1 molar ratio of insert to vector when taking into account the length of the DNA fragments as well as the DNA concentrations. The ligations reactions included 140 ng DNA vector (for a ˜5.0 kb vector), 1× T4 DNA ligase buffer, 2U T4 DNA ligase (Invitrogen), and DNA insert. After mixing the ligation reactions, they were incubated at room temperature for at least 3 hours and then electroporated into electro-competent TOP10F′ E. coli. The transformants were plated onto selective agar plates and grown up overnight in a 37° incubator. Individual colonies were picked and grown in 3.0 ml SB media overnight. On the following day, plasmid DNA was made from the bacterial pellet using the Qiagen miniprep kit.

DNA sequencing: When a recombinant clone at the endpoint of a cloning process showed the expected DNA band pattern in a diagnostic agarose gel, it was grown up in a large-scale bacterial culture, and maxi-prep plasmid DNA was made using the Qiagen Hi-Speed maxi-prep kit. The purified DNA was then quantitated with the Picogreen reagent. Following this, the DNA was sent along with the appropriate primer(s) (MWG Biotech) to either Retrogen (San Diego, Calif.) or MWG Biotech (High Point, N.C.) for DNA sequencing analysis and confirmation of the correct cloning result.

Generation of Stable Cell Lines Expressing Hy4-26A-IgG or Hy4-26C-IgG: 293 EBNA cells were transfected with the IgG expression vectors Hy426A-IgG or Hy426C-IgG, using the Effectene reagent (Qiagen; Valencia, Calif.). On day 3 post-transfection, the cells were split into 15 cm TC dishes at two different cell densities of approximately 1×10⁶ and 4×10⁶ per dish, and were subjected to two different puromycin concentrations of 5.0 or 20.0 mg/ml. From that point on, the cells were fed twice weekly with fresh media and the appropriate puromycin and G418 amounts. Two weeks after the 15 cm TC dishes were plated, they were examined for the presence of cell colonies that were of reasonable size, and spaced at least 1.0 cm apart to ensure clonal populations. The colonies were picked with a P200 pipettor under a confocal microscope and put into a 24 well plate with additional fresh media. The clones being selected at 20.0 g/ml puromycin were reduced to 10.0 μg/ml. Once the wells became confluent, the stable cell clones were transferred into a 12 well plate following to a T50 flask and finally into a T175 flask. Once the cells in a T175 flask became confluent, they were frozen down into 5 cryovials (1×10⁷ cells in 1 ml of 95% FBS and 10% DMSO).

ELISA screening of the stable cell clones: Hy426A-IgG and Hy426C stable cell line candidates (up to 10 clones each) were screened for functional expression of IgG. One frozen vial of each stable cell line was thawed into a separate T75 flask and the cells were allowed to attach overnight. The following morning, the media was changed with fresh media made with low-IgG FBS (Hyclone; Logan, Utah). The cells were allowed to grow for an additional two days at which point they were mostly confluent. An aliquot of the conditioned tissue culture media was then obtained for each stable cell clone. This media was examined in an ELISA assay on a plate coated overnight with the TC83 specific antigen and a BSA background control. Following the tissue culture media incubation in the ELISA plate, the bound IgG was detected using an AP-conjugate antibody specific to human LC (Peirce; Rockford, Ill.) and was developed with an alkaline phospatase substrate (Sigma; St. Louis, Mo.). The resulting data was graphed (FIG. 14). SDS-PAGE gels were run followed by Western blot analysis in order to verify expression of antibody light and heavy chains of expected sizes. Stable cell clones Hy426A-7, Hy426A-2, Hy426C-2, and Hy426C-6 were considered to be the best candidates for large-scale expression of the respective IgG.

Expression and Purification of Hy4-26A-IgG and Hy4-26C-IgG: The chosen stable cell clone from the ELISA expression analysis was immediately passed from the T75 flask into a T175 flask using the appropriate amount of puromycin selection and 500 μg/ml G418. This flask was allowed to grow for 2 days until it was completely confluent and was then passed 1:9 continuing with the selective media. Approximately 4 days later, these 9 flasks were again split 1:9 to make 81 flasks total. When the cells in these flasks reached about 90% confluency, the media was replaced with fresh low-IgG FBS media lacking the selective reagents. After two days of protein expression, the flasks were spiked with 20×“TC Sugar Rush” reagent (Harlow and Lane, 1988) consisting of 20% glucose and 0.5M HEPES. The cells were then allowed to incubate for 3 days more before harvesting the cell supernatants. The collected tissue culture (TC) media from these 81 flasks amounted to just over 2.0 Liters of conditioned TC media. This media was first spun in a centrifuge at 5000 rpm to pellet any cellular debris from the media. Following this, the media was concentrated 16× down to about 125 mls using a Pelicon concentrator device (Millipore; Billerica, Mass.) and finally passed through a 0.2 μM filter to prepare it for FPLC purification. The concentrated media supernatant was purified over an anti-human Fab column. The IgG was eluted from the column with 0.2M glycine, pH=2.2, and was collected as 1.5 ml fractions with neutralization using 2.0M Tris-Ci, pH=9.0 solution. The FPLC fractions were analyzed separately in a Coomassie-stained protein gel. The fractions containing significant amounts of IgG were combined and spun-dialyzed using a Centriprep YM30 (Millipore; Billerica, Mass.). The final centrifugation consisted of a concentration step to bring the IgG containing sample down to a more manageable volume of 2-3 mls. Before aliquoting the IgG sample into separate 0.5 ml fractions, it was quantitated using the BioRad protein quantitation kit (Hercules, Calif.) alongside an IgG standard. Additionally, the purified antibody was checked for specific activity in a TC83 titration ELISA assay consisting of antibody concentrations ranging from 5.0 to 0.005 μg/ml. A purified antibody preparation generally showed a good ELISA signal down at 0.05 μg/ml. Purified IgG aliquots were frozen at −20° C. until further use.

The above description sets forth preferred embodiments and examples. It should be understood that those skilled in the art will envision modifications of the embodiments and examples that, although not specifically stated herein, are still within the spirit and scope of any claims which may be appended hereto.

We claim:

1. A humanized antibody which binds to an epitope on an envelope glycoprotein of the Venezuelan equine encephalitis virus, the humanized antibody comprising at least one complementary determining region from a non-human antibody and at least one framework region from a human antibody.

2. A humanized antibody as in claim 1 which binds to the E2^{c} epitope of the major Venezuelan equine encephalitis virus glycoprotein E2.

3. A humanized antibody as in claim 1 comprising at least one complementary determining region from a murine antibody.

4. A humanized antibody as in claim 1 comprising CDR3 from a murine antibody.

5. A humanized antibody as in claim 1 comprising at least one complementary determining region from a murine antibody produced by hybridoma cells 3B4C-4.

6. A humanized antibody as in claim 1 comprising CDR3 from a murine antibody produced by hybridoma cells 3B4C-4.

7. A humanized antibody as in claim 1 comprising at least one complementary determining region that is a consensus sequence derived from a plurality of sequences of antibodies that bind to or are involved in infection by Venezuelan equine encephalitis virus.

8. A humanized antibody as in claim 1 comprising at least one framework region from a human antibody having at least 50% homology to the non-human antibody from which the at least one complementary determining region is derived.

9. A humanized antibody as in claim 1 comprising at least one framework region from a human germline antibody.

10. A humanized antibody as in claim 1 comprising a first framework region from a human germline antibody and a second framework region derived from a human re-arranged antibody.

11. An antibody fragment which binds to an epitope on an envelope glycoprotein of the Venezuelan equine encephalitis virus, the antibody fragment comprising at least one complementary determining region from a non-human antibody and at least one framework region from a human antibody.

12. An antibody fragment as in claim 11 which binds to the E2^{c} epitope of the major Venezuelan equine encephalitis virus glycoprotein E2.

13. An antibody fragment as in claim 1 comprising at least one complementary determining region from a murine antibody.

14. An antibody fragment as in claim 11 comprising CDR3 from a murine antibody.

15. An antibody fragment as in claim 11 comprising at least one complementary determining region from a murine antibody produced by hybridoma cells 3B4C-4.

16. An antibody fragment as in claim 11 comprising CDR3 from a murine antibody produced by hybridoma cells 3B4C-4.

17. An antibody fragment as in claim 11 comprising at least one complementary determining region that is a consensus sequence derived from a plurality of sequences of antibodies that bind to or are involved in infection by Venezuelan equine encephalitis virus.

18. An antibody fragment as in claim 11 comprising at least one framework region from a human antibody having at least 50% homology to the non-human antibody from which the at least one complementary determining region is derived.

19. An antibody fragment as in claim 11 comprising at least one framework region from a human germline antibody.

20. An antibody fragment as in claim 11 comprising a first framework region from a human germline antibody and a second framework region derived from a human re-arranged antibody.

21. A humanized antibody which binds to the E2^{c} epitope of the major Venezuelan equine encephalitis virus glycoprotein E2., the humanized antibody comprising CDR3 from a murine antibody produced by hybridoma cells 3B4C-4 and at least one framework region from a human antibody.

22. An antibody fragment which binds to the E2^{c} epitope of the major Venezuelan equine encephalitis virus glycoprotein E2., the antibody fragment comprising CDR3 from a murine antibody produced by hybridoma cells 3B4C-4 and at least one framework region from a human antibody.

23. An antibody comprising the heavy chain sequence of Sequence ID No. 98.

24. An antibody comprising the light chain sequence of Sequence ID No. 100.

24. An antibody comprising the light chain sequence of Sequence ID No. 102.

25. Nucleic acid encoding an antibody or antibody fragment in accordance with any of claims 1 through 24.

26. An expression vector comprising nucleic acid encoding an antibody or antibody fragment in accordance with any of claims 1 through 24.

27. A host cell transfected with an expression vector comprising nucleic acid encoding an antibody or antibody fragment in accordance with any of claims 1 through 24.

28. A pharmaceutical composition comprising an antibody or antibody fragment in accordance with any of claims 1 through 24 and a pharmaceutically acceptable carrier.

29. A method comprising administering to a subject an antibody or antibody fragment in accordance with any of claims 1 through 24.

30. A method comprising:
selecting at least one complementary determining region from a non-human antibody which binds to an epitope on an envelope glycoprotein of the Venezuelan equine encephalitis virus; and
combining the at least one non-human complementary determining region with at least one framework region from a human antibody to provide a humanized antibody.

31. A method as in claim 30 wherein the at least one complementary determining region from a non-human antibody which binds to an epitope on an envelope glycoprotein of the Venezuelan equine encephalitis virus binds to the E2^{c} epitope of the major Venezuelan equine encephalitis virus glycoprotein E2.

32. A method as in claim 30 wherein the at least one complementary determining region from a non-human antibody which binds to an epitope on an envelope glycoprotein of the Venezuelan equine encephalitis virus is derived from a murine antibody.

33. A method as in claim 30 wherein the at least one complementary determining region from a non-human antibody which binds to an epitope on an envelope glycoprotein of the Venezuelan equine encephalitis virus is a CDR3 from a murine antibody.

34. A method as in claim 30 wherein the at least one complementary determining region from a non-human antibody which binds to an epitope on an envelope glycoprotein of the Venezuelan equine encephalitis virus is derived from a murine antibody produced by hybridoma cells 3B4C-4.

35. A method as in claim 30 wherein the at least one complementary determining region from a non-human antibody which binds to an epitope on an envelope glycoprotein of the Venezuelan equine encephalitis virus is a CDR3 from a murine antibody produced by hybridoma cells 3B4C-4.

36. A method as in claim 30 wherein the at least one framework region is derived from a human antibody having at least 50% homology to the non-human antibody from which the at least one complementary determining region is derived.

37. A method as in claim 30 wherein the at least one framework region is derived from a human germline antibody.

38. A method comprising:
identifying nucleic acid encoding at least one complementary determining region from a non-human antibody which binds to an epitope on an envelope glycoprotein of the Venezuelan equine encephalitis virus; and
combining the nucleic acid encoding at least one non-human complementary determining region with nucleic acid encoding at least one framework region from a human antibody.

39. A method as in claim 38 wherein the nucleic acid encoding at least one complementary determining region from a non-human antibody which binds to an epitope on an envelope glycoprotein of the Venezuelan equine encephalitis virus is nucleic acid encoding at least one complementary determining region from a non-human antibody which binds to the E2^{c} epitope of the major Venezuelan equine encephalitis virus glycoprotein E2.

40. A method as in claim 38 wherein the nucleic acid encoding at least one complementary determining region from a non-human antibody which binds to an epitope on an envelope glycoprotein of the Venezuelan equine encephalitis virus is derived from a murine source.

41. A method as in claim 38 wherein the nucleic acid encoding at least one complementary determining region from a non-human antibody which binds to an epitope on an envelope glycoprotein of the Venezuelan equine encephalitis virus encodes a CDR3 from a murine antibody.

42. A method as in claim 38 wherein the nucleic acid encoding at least one complementary determining region from a non-human antibody which binds to an epitope on an envelope glycoprotein of the Venezuelan equine encephalitis virus encodes a complementary determining region from a murine antibody produced by hybridoma cells 3B4C-4.

43. A method as in claim 38 wherein the nucleic acid encoding at least one complementary determining region from a non-human antibody which binds to an epitope on an envelope glycoprotein of the Venezuelan equine encephalitis virus encodes a CDR3 from a murine antibody produced by hybridoma cells 3B4C-4.

44. A method as in claim 38 wherein the nucleic acid encoding at least one framework region encodes a human antibody having at least 50% homology to the non-human antibody from which the at least one complementary determining region is derived.

45. A method as in claim 38 wherein the nucleic acid encoding at least one framework region encodes at least one framework region derived from a human germline antibody.

46. A method as in claim 38 wherein the combined nucleic acid encodes a humanized antibody fragment.

47. A method as in claim 38 wherein the combined nucleic acid encodes a whole humanized antibody.

48. A method as in claim 38 further comprising the step of ligating the combined nucleic acid into an expression vector.

49. A method as in claim 48 further comprising the step of transfecting a host cell with the expression vector.

50. A cell line that expresses an antibody or antibody fragment in accordance with any of claims 1 through 24.
